(19) United States
(12) Patent Application Publication
(10) Pub. No.: US 2026/0078363 A1
(43) Pub. Date: Mar. 19, 2026

(54) RECOMBINANT CARBONIC ANHYDRASE POLYPEPTIDES AND METHODS OF USE THEREOF

(71) Applicants: Subha Kalyaanamoorthy, Waterloo (CA); Neha Saini, Kitchener (CA)

(72) Inventors: Subha Kalyaanamoorthy, Waterloo (CA); Neha Saini, Kitchener (CA)

(21) Appl. No.: 19/313,366

(22) Filed: Aug. 28, 2025

Related U.S. Application Data
(60) Provisional application No. 63/732,571, filed on Aug. 29, 2024.

(51) Int. Cl. C12N 9/88; B01D 53/14; B01D 53/62; B01D 53/84; C12N 15/52; C12N 15/63

(52) U.S. Cl. CPC C12N 9/88; B01D 53/1475; B01D 53/62; B01D 53/84; C12N 15/52; C12N 15/63; C12Y 402/01001; B01D 2257/504

(57) ABSTRACT

Disclosed herein are recombinant carbonic anhydrase polypeptide comprising an amino acid sequence having one or more amino acid modification as compared to SEQ ID NO: 13, wherein amino acid residues W32, Y34, G36, E37, G39, P40, W43, L46, E49, C53, K56, N57, Q58, P60, V61, A71, L73, L76, N79, Y80, 188, N90, N91, G92, H93, T94, V97, G109, L114, K115, Q116, F117, H118, F119, H120, A121, P122, S123, E124, G129, Y132, P133, E135, H137, V139, H140, D142, K143, D144, G145, N146, A148, V149, V152, F154, K155, E156, G157, N160, G175, N184, P190, Y195, Y196, S199, G200, D201, L202, T203, T24, P205, P206, C207, E209, G210, V211, W213, I214, V215, K217, S223, K224, Q226, I227, F230, M234, N239, R240, P241, Q243, P244, N246, R248, and/or I250, are unmodified, and variants thereof. Methods for use and kits are also disclosed herein.

### CROSS-REFERENCE TO RELATED APPLICATIONS

This application claims the benefit of priority to U.S. Provisional Application No. 63/732,571, filed Aug. 29, 2024, the contents of which is incorporated herein by reference in its entirety.

### INCORPORATION OF SEQUENCE LISTING

A computer readable form of the Sequence Listing “96547805_Sequence_Listing.xml” (67,808 bytes) created on Nov. 26, 2025, is herein incorporated by reference.

### FIELD

The present disclosure relates to recombinant carbonic anhydrase polypeptides, in particular, recombinant carbonic anhydrase polypeptides with improved thermostability and/or activity.

### BACKGROUND

The escalating emission of carbon dioxide (CO₂) due to prolonged fossil fuel consumption and its consequential impact on climate change underscore the urgent necessity for efficient CO₂ capture methods. Carbonic anhydrases (CAs), a group of ancient and fastest class of enzymes, catalyzing the reversible hydration of CO₂ to bicarbonate, have emerged as pivotal biocatalysts for CO₂ capture. However, the utilization of CAs in enhancing the carbon capture process is hampered by the natural enzymes' susceptibility to the harsh conditions prevalent in industrial settings. Hence, there is imperative need to design and engineer CA enzymes with high stability and catalytic performance.

### SUMMARY

Disclosed herein are recombinant carbonic anhydrases with improved thermostability and/or enhanced activity, generated through strategic evolution via the ancestral sequence reconstruction (ASR) technique. The recombinant carbonic anhydrases were successfully expressed in Escherichia coli and purified to homogeneity. It is demonstrated herein that generated recombinant carbonic anhydrases exhibit substantially augmented activity, e.g., a kcat of 4.3×10⁷ sec⁻¹, along with exceptional thermostability exceeding 100° C. Furthermore, the recombinant carbonic anhydrases described herein displayed reversible thermal denaturation, with the renatured enzyme retaining its catalytic functionality. Examination of temperature-dependent residual activities revealed that recombinant carbonic anhydrase node 7 maintains remarkable enzymatic activity even at 100° C., surpassing the performance of most natural CAs documented in the literature.

Accordingly, an aspect of the disclosure includes a recombinant carbonic anhydrase polypeptide comprising:
an amino acid sequence having one or more amino acid modification as compared to SEQ ID NO: 13, wherein amino acid residues W32, Y34, G36, E37, G39, P40, W43, L46, E49, C53, K56, N57, Q58, P60, V61, A71, L73, L76, N79, Y80, 188, N90, N91, G92, H93, T94, V97, G109, L114, K115, Q116, F117, H118, F119, H120, A121, P122, S123, E124, G129, Y132, P133, E135, H137, V139, H140, D142, K143, D144, G145, N146, A148, V149, V152, F154, K155, E156, G157, N160, G175, N184, P190, Y195, Y196, S199, G200, D201, L202, T203, T24, P205, P206, C207, E209, G210, V211, W213, I214, V215, K217, S223, K224, Q226, I227, F230, M234, N239, R240, P241, Q243, P244, N246, R248, and/or I250, are unmodified;
an amino acid sequence of SEQ ID NO: 1 or a sequence having at least about 55% sequence identity to SEQ ID NO: 1,
an amino acid sequence of SEQ ID NO: 2 or a sequence having at least about 57% sequence identity to SEQ ID NO: 2,
an amino acid sequence of SEQ ID NO: 3 or a sequence having at least about 65% sequence identity to SEQ ID NO: 3,
an amino acid sequence of SEQ ID NO: 4 or a sequence having at least about 67% sequence identity to SEQ ID NO: 4,
an amino acid sequence of SEQ ID NO: 5 or a sequence having at least about 76% sequence identity to SEQ ID NO: 5,
an amino acid sequence of SEQ ID NO: 6 or a sequence having at least about 69% sequence identity to SEQ ID NO: 6,
an amino acid sequence of SEQ ID NO: 7 or a sequence having at least about 73% sequence identity to SEQ ID NO: 7,
an amino acid sequence of SEQ ID NO: 8 or a sequence having at least about 86% sequence identity to SEQ ID NO: 8,
an amino acid sequence of SEQ ID NO: 9 or a sequence having at least about 87% sequence identity to SEQ ID NO: 9,
an amino acid sequence of SEQ ID NO: 10 or a sequence having at least about 93% sequence identity to SEQ ID NO: 10, and/or
an amino acid sequence of SEQ ID NO: 11 or a sequence having at least about 85% sequence identity to SEQ ID NO: 111.

An aspect of the disclosure includes a recombinant carbonic anhydrase polypeptide comprising an amino acid sequence having one or more amino acid modification as compared to SEQ ID NO: 13, wherein amino acid residues W32, Y34, G36, E37, G39, P40, W43, L46, E49, C53, K56, N57, Q58, P60, V61, A71, L73, L76, N79, Y80, 188, N90, N91, G92, H93, T94, V97, G109, L114, K115, Q116, F117, H118, F119, H120, A121, P122, S123, E124, G129, Y132, P133, E135, H137, V139, H140, D142, K143, D144, G145, N146, A148, V149, V152, F154, K155, E156, G157, N160, G175, N184, P190, Y195, Y196, S199, G200, D201, L202, T203, T24, P205, P206, C207, E209, G210, V211, W213, I214, V215, K217, S223, K224, Q226, I227, F230, M234, N239, R240, P241, Q243, P244, N246, R248, and/or I250, are unmodified.

An aspect of the disclosure includes a recombinant carbonic anhydrase polypeptide comprising:
an amino acid sequence of SEQ ID NO: 1 or a sequence having at least about 55% sequence identity to SEQ ID NO: 1;
an amino acid sequence of SEQ ID NO: 2 or a sequence having at least about 57% sequence identity to SEQ ID NO: 2;
an amino acid sequence of SEQ ID NO: 3 or a sequence having at least about 65% sequence identity to SEQ ID NO: 3;
an amino acid sequence of SEQ ID NO: 4 or a sequence having at least about 67% sequence identity to SEQ ID NO: 4;
an amino acid sequence of SEQ ID NO: 5 or a sequence having at least about 76% sequence identity to SEQ ID NO: 5;
an amino acid sequence of SEQ ID NO: 6 or a sequence having at least about 69% sequence identity to SEQ ID NO: 6;
an amino acid sequence of SEQ ID NO: 7 or a sequence having at least about 73% sequence identity to SEQ ID NO: 7;
an amino acid sequence of SEQ ID NO: 8 or a sequence having at least about 86% sequence identity to SEQ ID NO: 8;
an amino acid sequence of SEQ ID NO: 9 or a sequence having at least about 87% sequence identity to SEQ ID NO: 9;
an amino acid sequence of SEQ ID NO: 10 or a sequence having at least about 93% sequence identity to SEQ ID NO: 10; or
an amino acid sequence of SEQ ID NO: 11 or a sequence having at least about 85% sequence identity to SEQ ID NO: 11.

Another aspect of the disclosure includes a nucleic acid encoding the recombinant carbonic anhydrase polypeptide described herein.

Another aspect of the disclosures includes a vector or expression cassette comprising encoding a recombinant carbonic anhydrase polypeptide described herein or the isolated nucleic acid described herein. In some embodiments, the vector is a pET-28(a) expression vector.

An aspect of the disclosure includes a cell comprising the vector or expression cassette described herein. In some embodiments, the cell is an isolated cell. In some embodiments, the cell is an E. coli cell. In some embodiments, the cell expresses a recombinant carbonic anhydrase polypeptide described herein. In some embodiments, the cell is an isolated cell.

Another aspect of the disclosure includes a composition comprising a recombinant carbonic anhydrase polypeptide described herein, an isolated nucleic acid described herein, a vector or expression cassette described herein, and/or a cell described herein, and optionally a diluent or carrier.

An aspect of the disclosure is a method of making a recombinant carbonic anhydrase polypeptide described herein, the method comprising culturing a host cell under conditions enabling the expression of the recombinant, carbonic anhydrase polypeptide, and recovering the recombinant carbonic anhydrase polypeptide.

An aspect of the disclosure includes a use of the above mentioned recombinant carbonic anhydrase polypeptides the nucleic acid defined herein, the vector or expression cassette described herein, the cell described herein, and/or the composition described herein, in an industrial process for capturing CO₂ from a CO₂-containing effluent or gas.

An aspect of the disclosure includes a recombinant polypeptide described herein, the nucleic acid defined herein, the vector or expression cassette described herein, the cell described herein, and/or the composition described herein, for use in capturing CO₂, optionally converting CO₂ into bicarbonate and H+ ions.

An aspect of the disclosure includes use of a recombinant polypeptide described herein, the nucleic acid defined herein, the vector or expression cassette described herein, the cell described herein, and/or the composition described herein, for capturing CO₂, optionally converting CO₂ into bicarbonate and H+ ions.

An aspect described herein is a method for absorbing or capturing CO₂ from a CO₂-containing effluent or gas, the process comprising: contacting the CO₂-containing effluent or gas with an aqueous absorption solution to dissolve the CO₂ into the aqueous absorption solution; and providing the recombinant carbonic anhydrase polypeptide defined herein, the nucleic acid defined herein, the vector or expression cassette described herein, the cell described herein, and/or the composition described herein, to catalyze the hydration reaction of the dissolved CO₂ into bicarbonate and hydrogen ions or the reverse reaction.

An aspect of the disclosures includes a method for removing carbon dioxide from a gas stream comprising the step of contacting the gas stream with a composition comprising a recombinant carbonic anhydrase polypeptide described herein, the nucleic acid defined herein, the vector or expression cassette described herein, the cell described herein, and/or the composition described herein, whereby carbon dioxide from the gas stream is dissolved in the solution and converted to hydrated carbon dioxide.

An aspect of the disclosure includes a method of identifying recombinant carbonic anhydrase polypeptides, optionally with improved thermostability and/or activity, e.g., hydratase and/or esterase activity using ancestral sequence reconstruction methods described e.g., in the Examples.

An aspect of the disclosures includes a kit comprising one or more recombinant carbonic anhydrase polypeptides described herein, an isolated nucleic acid described herein, the vector or ex-pression cassette described herein, the host cell described herein, and/or a composition de-scribed herein, and optionally instructions for use. In some embodiments, the kit further comprises one or more vial or tube. In some embodiments, the kit is for use in one or more of the methods and/or uses described herein.

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages objects and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### BRIEF DESCRIPTION OF THE DRAWINGS

Further objects, features and advantages of the disclosure will become apparent from the following detailed description taken in conjunction with the accompanying figures showing illustrative embodiments of the disclosure, in which:

FIG. 1. Ancestral Sequence reconstruction (ASR). Phylogenetic tree constructed for SazCA, to infer the common ancestral sequences. The tree was inferred using maximum likelihood method and configured in FigTree. Further the tree was edited using Itol.

FIGS. 2A-B. A. Size exclusion Chromatography (SEC) profile for AncCA and SazCA. His-Tag purified enzymes were loaded onto the Hiload 16/600 200 pg Superdex column in buffer containing 50 mM Tris-SO4, 300 mM NaCl pH 8.2 with the flow rate of 0.8 ml/min. A. Plot for absorbance at 280 nm versus elution volume for AncCA and SazCA. B. SDS-PAGE gel of dAncCA and mAncCA. For reference the molecular weight of AncCA is ˜27 kDa.

FIGS. 3A-C. Differential scanning calorimetry (DSC) thermogram of unfolding transition for A. dAncCA and B. SazCA. Continuous curves show the best fit to a non-two-state model and the dotted curve shows the buffer-subtracted, baseline corrected raw data for DSC. C. Reversibility of thermal unfolding (scan 2) for dAncCA. Raw data and fitted data (solid and dashed line, respectively) are shown.

FIGS. 4A-B. Intrinsic Trp fluorescence spectra for A. dAncCA and B. mAncCA obtained at temperatures ranging from 20° C.-100° C. The curve labelled refold 20° C. represents reversible scan after cooling to 20° C.

FIGS. 5A-C. far-UV CD spectra monitored at different temperatures for A. dAncCA B. mAncCA and C. SazCA. Data was acquired from 20° C.-100° C. to monitor structural unfolding and black dotted line (refold 20° C.) corresponds to reversible scan after cooling the instrument at 20° C.

FIGS. 6A-C: Enzymatic activity using CO₂ and pNPA as substrate. Residual hydratase activities for dAncCA (square), mAncCA (circle) and SazCA (triangle) for A. Short-term (30 min) incubation and B. long-term (180 min) incubation at various temperatures. C. Esterase activity assay for Short-term (30 min) incubation at various temperatures between 20° C.-100° C. The residual activities were estimated relative to the activities at 100% that correspond to the activity of untreated samples for each enzyme. Each value represents the mean of experiments done in triplicate and the error bars represent the standard deviation.

FIGS. 7A-C: Effect of repeated heating-cooling cycles on oligomeric state and hydratase activity: repeated heat-cool experiments on both dAncCA and mAncCA were performed and the results were compared to non-heat-treated samples. During single heat-cool cycle (cycle 1), the enzymes were heated to 100° C. and cooled on ice. In double heat-cool cycle (cycle 2) the enzyme was heated and cooled twice to analyze the effect of multiple unfolding and refolding on the oligomeric state and activity. A. SEC for dAncCA was performed for all three samples; non-heated enzymes (no heat), cycle 1 and cycle 2 on Superdex™ 75 10/300 column with a flow rate of 0.3 mL/min. Peak at ˜10 mL and ˜12 mL corresponds to dimer and monomer fraction respectively. B. percentage residual hydratase activity for dAncCA cycle 1 and cycle 2 relative to 100% activity corresponding to no heat sample. C. percentage residual hydratase activity for mAncCA cycle 1 and cycle 2 relative to 100% activity corresponding to no heat sample.

FIGS. 8A-B: Enzymatic activity using CO₂ as a substrate: hydratase activity at different temperatures at 30 minutes and 180 minutes for node 5 dimer (A) and monomer (B). Residual activities for Node5_dimer (A) and Node5_monomer (B) for Short-term (30 min) incubation and long-term (180 min) incubation at various temperatures between 20° C. and 100° C. The residual activities were estimated relative to the activities at 100% that correspond to the activity of untreated samples for each enzyme.

FIGS. 9A-B: Enzymatic activity using CO₂ as a substrate: hydratase activity at different temperatures at 30 minutes and 180 minutes for node 6 dimer (A) and monomer (B). Residual activities for Node6_dimer (A) and Node6_monomer (B) for Short-term (30 min) incubation and long-term (180 min) incubation at various temperatures between 20° C. and 100° C. The residual activities were estimated relative to the activities at 100% that correspond to the activity of untreated samples for each enzyme.

FIG. 10: Differential scanning calorimetry (DSC) thermogram for node5 dimer. The continuous curve represents the buffer-subtracted, baseline-corrected raw data for DSC. The data was fit to a non-two transition state model, and each peak represents a different transition state the model was fit to transition state 1: 68° C., transition state 2: 78° C., transition state 3: 100° C. and transition state 4: 107° C.

FIG. 11: Differential scanning calorimetry (DSC) thermogram for node6 dimer. The continuous curve represents the buffer-subtracted, baseline-corrected raw data for DSC. The data was fit to a non-two transition state model, and each peak represents a different transition state the model was fit to transition state 1: 68° C., transition state 2: 76° C. and transition state 3: 105° C.

FIGS. 12A-B depict the esterase activity after incubation for 30 minutes and 180 minutes at different temperatures from 20° C. to 100° C. for node 5 dimer (A) and monomer (B) that were measured using p-NPA substrate. The residual activities were estimated relative to the activities at 100% that correspond to the activity of untreated samples for each enzyme.

FIGS. 13A-B depict the esterase activity after incubation for 30 minutes and 180 minutes at different temperatures from 20° C. to 100° C. for node 6 dimer (A) and monomer (B) that were measured using p-NPA as a substrate. The residual activities were estimated relative to the activities at 100% that correspond to the activity of untreated samples for each enzyme.

FIG. 14 is a schematic representation of the pET-28a(+) vector and corresponding nucleotide sequence and related amino acid sequence.

### DETAILED DESCRIPTION OF VARIOUS EMBODIMENTS

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

The following non-limiting examples are illustrative of the present application:

### I. Definitions

As used herein, the following terms may have meanings ascribed to them below, unless specified otherwise. However, it should be understood that other meanings that are known or understood by those having ordinary skill in the art are also possible, and within the scope of the present disclosure. In the case of conflict, the present specification, including definitions, will control. In addition, the materials, methods, and examples are illustrative only and not intended to be limiting.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either both of those included limits are also included in the description.

The term “about” as used herein may be used to take into account experimental error and variations that would be expected by a person having ordinary skill in the art. For example, “about” may mean plus or minus 10%, or plus or minus 5%, of the indicated value to which reference is being made.

As used herein the singular forms “a”, “an”, and “the” include plural references unless the context clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein, the expression “recombinant carbonic anhydrase polypeptide(s)” refers to non-naturally occurring enzymes capable of catalyzing the hydration of carbon dioxide engineered or produced using recombinant technology. In some embodiments, the recombinant carbonic anhydrase polypeptides described herein may comprise any type of modification (e.g., chemical or post-translational modifications such as acetylation, phosphorylation, glycosylation, sulfatation, sumoylation, prenylation, ubiquitination, etc.). For further clarity, polypeptide modifications are envisaged so long as the modification does not destroy the carbonic anhydrase activity of the carbonic anhydrase polypeptides described herein.

As used herein, the term “wild type SazCa” refers to non-modified or naturally occurring Sulfurihydrogenibium azorense, e.g., as described in accession number ACN99362 or the sequence as defined in SEQ ID NO: 13.

As used herein, the term “unmodified” refers to nucleotides or amino acid residues of a polynucleotide sequence or polypeptide sequence, respectively, that are those that are unaltered in a sequence (e.g., in the same position of two or more sequences being compared).

As used herein, the term “modification” refers to nucleotides or amino acid residues of a polynucleotide sequence or polypeptide sequence, respectively, that are those that are altered or different in a sequence (e.g., in the same position of two or more sequences being compared).

Accordingly, an aspect of the disclosure includes a recombinant carbonic anhydrase polypeptide comprising an amino acid sequence having one or more amino acid modification as compared to SEQ ID NO: 13, wherein amino acid residues W32, Y34, G36, E37, G39, P40, W43, L46, E49, C53, K56, N57, Q58, P60, V61, A71, L73, L76, N79, Y80, 188, N90, N91, G92, H93, T94, V97, G109, L114, K115, Q116, F117, H118, F119, H120, A121, P122, S123, E124, G129, Y132, P133, E135, H137, V139, H140, D142, K143, D144, G145, N146, A148, V149, V152, F154, K155, E156, G157, N160, G175, N184, P190, Y195, Y196, S199, G200, D201, L202, T203, T24, P205, P206, C207, E209, G210, V211, W213, I214, V215, K217, S223, K224, Q226, I227, F230, M234, N239, R240, P241, Q243, P244, N246, R248, and/or I250, are unmodified. For example, these amino acid residues are unmodified or conserved when the amino acid sequences of nodes 1-11 (SEQ ID NOs: 1-11) and SazCA (SEQ ID NO: 13) are aligned (e.g., see Table 5 alignment).

In some embodiments, the recombinant carbonic anhydrase polypeptide comprises an amino acid sequence having at least about 45% sequence identity to SEQ ID NO: 13. In some embodiments, the recombinant carbonic anhydrase polypeptide comprises an amino acid sequence having at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 13.

In some embodiments, the recombinant carbonic anhydrase polypeptide comprises an amino acid sequence having at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% sequence identity to any one of SEQ ID NOs: 1-11.

An aspect of the disclosure includes a recombinant carbonic anhydrase polypeptide comprising:
an amino acid sequence of SEQ ID NO: 1 or a sequence having at least about 55% sequence identity to SEQ ID NO: 1;
an amino acid sequence of SEQ ID NO: 2 or a sequence having at least about 57% sequence identity to SEQ ID NO: 2;
an amino acid sequence of SEQ ID NO: 3 or a sequence having at least about 65% sequence identity to SEQ ID NO: 3;
an amino acid sequence of SEQ ID NO: 4 or a sequence having at least about 67% sequence identity to SEQ ID NO: 4;
an amino acid sequence of SEQ ID NO: 5 or a sequence having at least about 76% sequence identity to SEQ ID NO: 5;
an amino acid sequence of SEQ ID NO: 6 or a sequence having at least about 69% sequence identity to SEQ ID NO: 6;
an amino acid sequence of SEQ ID NO: 7 or a sequence having at least about 73% sequence identity to SEQ ID NO: 7;
an amino acid sequence of SEQ ID NO: 8 or a sequence having at least about 86% sequence identity to SEQ ID NO: 8;
an amino acid sequence of SEQ ID NO: 9 or a sequence having at least about 87% sequence identity to SEQ ID NO: 9;
an amino acid sequence of SEQ ID NO: 10 or a sequence having at least about 93% sequence identity to SEQ ID NO: 10; and/or an amino acid sequence of SEQ ID NO: 11 or a sequence having at least about 85% sequence identity to SEQ ID NO: 11.

In some embodiments, the recombinant carbonic anhydrase polypeptide comprises an amino acid sequence having at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1.

In some embodiments, the recombinant carbonic anhydrase polypeptide comprises an amino acid sequence at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 2.

In some embodiments, the recombinant carbonic anhydrase polypeptide comprises an amino acid sequence having at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 3.

In some embodiments, the recombinant carbonic anhydrase polypeptide comprises an amino acid sequence having at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 4.

In some embodiments, the recombinant carbonic anhydrase polypeptide comprises an amino acid sequence having at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 5.

In some embodiments, the recombinant carbonic anhydrase polypeptide comprises an amino acid sequence having at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 6.

In some embodiments, the recombinant carbonic anhydrase polypeptide comprises an amino acid sequence having at least about 84% sequence identity to SEQ ID NO: 6. For example Node 7 (SEQ ID NO: 7) comprises an amino acid sequence having about 84% sequence identity to SEQ ID NO: 6 and has improved thermostability and activity as compared to wild type SazCA, as described herein.

In some embodiments, the recombinant carbonic anhydrase polypeptide comprises an amino acid sequence having at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 7.

In some embodiments, the recombinant carbonic anhydrase polypeptide comprises an amino acid sequence having at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 8.

In some embodiments, the recombinant carbonic anhydrase polypeptide comprises an amino acid sequence having at least about 67%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 9.

In some embodiments, the recombinant carbonic anhydrase polypeptide comprises an amino acid sequence having at least about 93%, at least about 94%, at least about 95%, at least about 96%, at least about 97%, at least about 98%, at least about 99%, or at least about 99.5% sequence identity to SEQ ID NO: 10.

In some embodiments, the recombinant carbonic anhydrase polypeptide comprises an amino acid sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 11.

For example, Node 7 (SEQ ID NO: 7) comprises an amino acid sequence having about 57% sequence identity to SEQ ID NO: 1, about 58% sequence identity to SEQ ID NO: 2, about 69% sequence identity to SEQ ID NO: 3, about 72% sequence identity to SEQ ID NO: 4, about 74% sequence identity to SEQ ID NO: 5, about 84% sequence identity to SEQ ID NO: 6, about 89% sequence identity to SEQ ID NO: 8, about 63% sequence identity to SEQ ID NO: 9, about 62% sequence identity to SEQ ID NO: 10, about 62% sequence identity to SEQ ID NO: 11, and has improved thermostability and activity as compared to wild type SazCA, as described herein.

For example, Node 6 (SEQ ID NO: 6) comprises an amino acid sequence having about 63% sequence identity to SEQ ID NO: 1, about 66% sequence identity to SEQ ID NO: 2, about 77% sequence identity to SEQ ID NO: 3, about 82% sequence identity to SEQ ID NO: 4, about 89% sequence identity to SEQ ID NO: 5, about 84% sequence identity to SEQ ID NO: 7, about 75% sequence identity to SEQ ID NO: 8, about 76% sequence identity to SEQ ID NO: 9, about 72% sequence identity to SEQ ID NO: 10, about 75% sequence identity to SEQ ID NO: 11, and has improved thermostability as compared to wild type SazCA, as described herein.

For example, Node 5 (SEQ ID NO: 5) comprises an amino acid sequence having about 68% sequence identity to SEQ ID NO: 1, about 71% sequence identity to SEQ ID NO: 2, about 85% sequence identity to SEQ ID NO: 3, about 88% sequence identity to SEQ ID NO: 4, about 89% sequence identity to SEQ ID NO: 6, about 74% sequence identity to SEQ ID NO: 7, about 67% sequence identity to SEQ ID NO: 8, about 84% sequence identity to SEQ ID NO: 9, about 80% sequence identity to SEQ ID NO: 10, about 84% sequence identity to SEQ ID NO: 11, and has improved thermostability as compared to wild type SazCA, as described herein.

In some embodiments, the recombinant carbonic anhydrase polypeptide comprises amino acid residues: W2, Y4, G6, E7, G9, P10, W13, L16, E19, C23, K26, N27, Q28, P30, V31, A39, L41, L44, N47, Y48, 155, N57, N58, G59, H60, T61, V64, G76, L81, K82, Q83, F84, H85, F86, H87, A88, P89, S90, E91, G96, Y99, P100, E102, H104, V106, H107, D109, K110, D111, G112, N113, A115, V116, V119, F121, K122, E123, G124, N127, G142, N151, P157, Y162, Y163, S166, G167, D168, L169, T170, T171, P172, P173, C174, E176, G177, V178, W180, I181, V182, K184, S190, K191, Q193, I194, F197, M201, N206, R207, P208, Q210, P211, N213, R215, and/or I217.

In some embodiments, the recombinant carbonic anhydrase polypeptide comprises an amino acid sequence of:

(SEQ ID NO: 12)
X₁WX₂YX₃GEX₄GPX₅X₆WX₇X₈LX₉X₁₀EX₁₁X₁₂X_{13C}X₁₄X₁₅
KNQX₁₆PVX₁₇X₁₈X₁₉X₂₀X₂₁X₂₂X₂₃AX₂₄LX₂₅X₂₆LX₂₇
X₂₈NYX₂₉X₃₀X₃₁X₃₂X₃₃X₃₄IX₃₅NNGHTX₃₆X₃₇VX₃₈X₃₉
X₄₀X₄₁X₄₂X₄₃X₄₄X₄₅X₄₆X₄₇X₄₈GX₄₉X₅₀X₅₁X₅₂
LKQFHFHAPSEX₅₃X₅₄X₅₅X₅₆GX₅₇X₅₈YPX₅₉EX₆₀HX₆₁
VHX₆₂DKDGNX₆₃AVX₆₄X₆₅VX₆₆FKEGX₆₇X₆₈NX₆₉X₇₀X₇₁
X₇₂X₇₃X₇₄X₇₅X₇₆X₇₇X₇₈X₇₉X₈₀X₈₁X₈₂GX₈₃X₈₄X₈₅X₈₆
X₈₇X₈₈X₈₉X₉₀NX₉₁X₉₂X₉₃X₉₄X₉₅PX₉₆X₉₇X₉₈X₉₉
YYX₁₀₀X₁₀₁SGSLTTPPCX₁₀₂EGVX₁₀₃WIVX₁₀₄KX₁₀₅X₁₀₆
X₁₀₇X₁₀₈X₁₀₉SKX₁₁₀QIX₁₁₁X₁₁₂FX₁₁₃X₁₁₄X₁₁₅MX₁₁₆
X₁₁₇X₁₁₈X₁₁₉NRPX₁₂₀QPX₁₂₁NX₁₂₂RX₁₂₃IX₁₂₄X₁₂₅,

wherein: X₁ is H or N; X₂ is G or S; X₃ is S, H or E; X₄ is T or N; X₅ is E or Q; X₆ is N or H; X₇ is A or G; X₈ is K or D; X₉ is T, N or K; X₁₀ is P, N or D; X₁₁ is F or Y; X₁₂ is G, F, I or A; X₁₃ is A, W or M; X₁₄ is A, N or K; X₁₅ is G or L; X₁₆ is T or S; X₁₇ is N or D; X₁₈ is L or I; X₁₉ is S, D or N; X₂₀ is G, D or R; X₂₁ is F, K, I or T; X₂₂ is V or I; X₂₃ is K, H or E; X₂₄ is E or K; X₂₅ is K or E; X₂₆ is P, K, or D; X₂₇ is K or N; X₂₈ is F or I; X₂₉ is K, N or S; X₃₀ is A, K or S; X₃₁ is G or A; X₃₂ is G, N or A; X₃₃ is S, T or P; X₃₄ is Q, E or S; X₃₅ is L, V, or T; X₃₆ is V or I; X₃₇ is Q or K; X₃₈ is V, N or S; X₃₉ is Y or V; X₄₀ is D, L, K, E or A; X₄₁ is A, E or P; X₄₂ is G or D; X₄₃ is S, F, or N; X₄₄ is N, K, T or Y; X₄₅ is V, L or I; X₄₆ is V or N; X₄₇ is I or V; X₄₈ is D or K; X₄₉ is V, K, I or T; X₅₀ is E, K or R; X₅₁ is Y or F; X₅₂ is A, H or E; X₅₃ is H or N; X₅₄ is Q, T or K; X₅₅ is I or V; X₅₆ is K, N or E; X₅₇ is E, K, or Q; X₅₈ is S or Y; X₅₉ is L or F; X₆₀ is G, M or A; X₆₁ is F or L; X₆₂ is A or K; X₆₃ is L or I; X₆₄ is V or I; X₆₅ is T or G; X₆₆ is M, F, I or V; X₆₇ is H or K; X₆₈ is E, K or A; X₆₉ is E, P, or Q; X₇₀ is A, E or V; X₇₁ is L or 1; X₇₂ is E or D; X₇₃ is S or K; X₇₄ is L, V or I; X₇₅ is W or F; X₇₆ is A, T or K; X₇₇ is H or N; X₇₈ is M, A or L; X₇₉ is P or L; X₈₀ is A, K or S; X₈₁ is K or E; X₈₂ is E, A or V; X₈₃ is D, S, E or K; X₈₄ is K or T; X₈₅ is I or V; X₈₆ is L or F; X₈₇ is S, D, or A; X₈₈ is P, G, H or S; X₈₉ is A, S or K; X₉₀ is F, N or I; X₉₁ is A, L, or M; X₉₂ is L, N or Y; X₉₃ is K, A or D; X₉₄ is L or F; X₉₅ is F or L; X₉₆ is K or P; X₉₇ is N, D, V or K; X₉₈ is H or K; X₉₉ is E, A, N, K or D; X₁₀₀ is R or T; X₁₀₁ is F or Y; X₁₀₂ is T or S; X₁₀₃ is R or L; X₁₀₄ is M or L; X₁₀₅ is K, Q, or E; X₁₀₆ is P or E; X₁₀₇ is V, I, M or L; X₁₀₈ is F, T, E or S; X₁₀₉ is V, A, L or M; X₁₁₀ is A, Q or E; X₁₁₁ is D or E; X₁₁₂ is A, L or K; X₁₁₃ is K or R; X₁₁₄ is K or S; X₁₁₅ is V, I or L; X₁₁₆ is G, V or K; X₁₁₇ is H or G; X₁₁₃ is D, A, or N; X₁₁₉ is N or T; X₁₂₀ is L, T, or V; X₁₂₁ is I or L; X₁₂₂ is A or S; X₁₂₃ is E, Y, M, V, or T; X₁₂₄ is L or M; and/or X₁₂₅ is E or G.

In some embodiments, the amino acid sequence comprises or consists of any one of SEQ ID NOs: 1-11. In some embodiments, the amino acid sequence comprises or consists of SEQ ID NO: 1. In some embodiments, the amino acid sequence comprises or consists of SEQ ID NO: 2. In some embodiments, the amino acid sequence comprises or consists of SEQ ID NO: 3. In some embodiments, the amino acid sequence comprises or consists of SEQ ID NO: 4. In some embodiments, the amino acid sequence comprises or consists of SEQ ID NO: 5. In some embodiments, the amino acid sequence comprises or consists of SEQ ID NO: 6. In some embodiments, the amino acid sequence comprises or consists of SEQ ID NO: 7. In some embodiments, the amino acid sequence comprises or consists of SEQ ID NO: 8. In some embodiments, the amino acid sequence comprises or consists of SEQ ID NO: 9. In some embodiments, the amino acid sequence comprises or consists of SEQ ID NO: 10. In some embodiments, the amino acid sequence comprises or consists of SEQ ID NO: 11.

In some embodiments, the amino acid sequence comprises or consists of any one of SEQ ID NOs: 5-7. In some embodiments, the amino acid sequence comprises or consists of SEQ ID NO: 5. In some embodiments, the amino acid sequence comprises or consists of SEQ ID NO: 6. In some embodiments, the amino acid sequence comprises or consists of SEQ ID NO: 7.

In some embodiments, the recombinant carbonic anhydrase polypeptide is a fusion polypeptide. In some embodiments, the recombinant carbonic anhydrase polypeptide is a fusion polypeptide comprising the amino acid sequence of at least one of any of the recombinant carbonic anhydrase polypeptides described herein. In some embodiments, the recombinant carbonic anhydrase polypeptide is a fusion polypeptide comprising the amino acid sequence of at least two of any of the recombinant carbonic anhydrase polypeptides described herein. In some embodiments, the recombinant carbonic anhydrase polypeptide is a fusion polypeptide comprising the amino acid sequence of at least two of SEQ ID NOs: 1-11. In some embodiments, the recombinant carbonic anhydrase polypeptide is a fusion polypeptide comprising the amino acid sequence of at least one of SEQ ID NOs: 1-11.

In some embodiments, the recombinant carbonic anhydrase polypeptide has carbonic anhydrase activity. In some embodiments, the recombinant carbonic anhydrase polypeptide has hydratase and/or esterase activity. In some embodiments, the recombinant carbonic anhydrase polypeptide catalyzes the reversible hydration of CO₂ to bicarbonate and protons.

In some embodiments, the recombinant carbonic anhydrase polypeptide comprises increased activity as compared to wild type SazCA.

In some embodiments, the recombinant carbonic anhydrase polypeptide comprises increased hydratase activity as compared to wild type SazCA.

In some embodiments, the recombinant carbonic anhydrase polypeptide comprises at least about 2-fold higher hydratase activity as compared to wild type SazCA.

In some embodiments, the recombinant carbonic anhydrase polypeptide comprises increased thermostability as compared to wild type SazCA. For example, the recombinant carbonic anhydrase polypeptide with increased thermostability as compared to wild type SazCA may experience protein unfolding at higher temperatures than wild type SazCA. The recombinant carbonic anhydrase with increased thermostability as compared to wild type SazCA may retain higher levels of activity, e.g., hydratase and/or esterase activity, than wild type SazCA at higher temperatures e.g., for short or extended periods of time. For example, the recombinant carbonic anhydrase polypeptide with increased thermostability as compared to wild type SazCA may retain more than about 10% activity, e.g., at least about 20% activity, at least about 25% activity, at least about 30% activity, at least about 35% activity, at least about 40% activity, at least about 45% activity, at least about 50% activity, at least about 55% activity, at least about 60% activity, at least about 65% activity, at least about 70% activity, at least about 75% activity, at least about 80% activity, at least about 85% activity, at least about 90% activity, at least about 95% activity, at least about 100% activity. For example, a short period of exposure to high temperatures may include temperatures up to at least about 100° C. for up to about 30 minutes. A long period of exposure to high temperatures may include for example, temperatures up to at least about 100° C. for up to about 180 minutes.

In some embodiments, the recombinant carbonic anhydrase polypeptide comprises at least about 10° C.-15° C. increase in its temperature for unfolding as compared to wild type SazCA. In some embodiments, the recombinant carbonic anhydrase polypeptide comprises at least about 10° C. increase in its temperature for unfolding as compared to wild type SazCA. In some embodiments, the recombinant carbonic anhydrase polypeptide comprises at least about 10.5° C. increase in its temperature for unfolding as compared to wild type SazCA. In some embodiments, the recombinant carbonic anhydrase polypeptide comprises at least about 11° C. increase in its temperature for unfolding as compared to wild type SazCA. In some embodiments, the recombinant carbonic anhydrase polypeptide comprises at least about 11.5° C. increase in its temperature for unfolding as compared to wild type SazCA. In some embodiments, the recombinant carbonic anhydrase polypeptide comprises at least about 12° C. increase in its temperature for unfolding as compared to wild type SazCA. In some embodiments, the recombinant carbonic anhydrase polypeptide comprises at least about 12.5° C. increase in its temperature for unfolding as compared to wild type SazCA. In some embodiments, the recombinant carbonic anhydrase polypeptide comprises at least about 13° C. increase in its temperature for unfolding as compared to wild type SazCA. In some embodiments, the recombinant carbonic anhydrase polypeptide comprises at least about 13.5° C. increase in its temperature for unfolding as compared to wild type SazCA. In some embodiments, the recombinant carbonic anhydrase polypeptide comprises at least about 14° C. increase in its temperature for unfolding as compared to wild type SazCA. In some embodiments, the recombinant carbonic anhydrase polypeptide comprises at least about 14.5° C. increase in its temperature for unfolding as compared to wild type SazCA. In some embodiments, the recombinant carbonic anhydrase polypeptide comprises at least about 15° C. increase in its temperature for unfolding as compared to wild type SazCA.

In some embodiments, the recombinant carbonic anhydrase polypeptide renatures after denaturation. For example, when the recombinant carbonic anhydrase polypeptide is exposed to high temperatures e.g., 100° C., once the temperature is reduced e.g. to 20° C., it can renature while retaining enzymatic activity e.g., hydratase and/or esterase activity.

In some embodiments, the recombinant carbonic anhydrase polypeptide comprises an increased maximum reaction rate (Vmax) for CO₂ hydration as compared to wild type SazCA. In some embodiments, the recombinant carbonic anhydrase polypeptide comprises a Vmax about 3-fold times higher than SazCA. In some embodiments, the recombinant carbonic anhydrase polypeptide comprises at least about 1-fold higher turnover number for CO₂ hydration as compared to wild type SazCA.

In some embodiments, the recombinant carbonic anhydrase polypeptide comprises at least about 4-fold higher catalytic efficiency as compared to wild type SazCA. In some embodiments, the recombinant carbonic anhydrase polypeptide comprises at least about 3-fold higher catalytic efficiency as compared to wild type SazCA. In some embodiments, the recombinant carbonic anhydrase polypeptide comprises at least about 2-fold higher catalytic efficiency as compared to wild type SazCA. In some embodiments, the recombinant carbonic anhydrase polypeptide comprises at least about 1-fold higher catalytic efficiency as compared to wild type SazCA.

In some embodiments, the recombinant carbonic anhydrase polypeptide is a monomer. In some embodiments, the recombinant carbonic anhydrase polypeptide is a dimer.

In some embodiments, the recombinant carbonic anhydrase polypeptide is a purified polypeptide. In some embodiments, the recombinant carbonic anhydrase polypeptide is an isolated polypeptide.

Another aspect of the disclosure includes a nucleic acid encoding the recombinant carbonic anhydrase polypeptide described herein.

In some embodiments, the nucleic acid comprises or consists of the nucleotide sequence of SEQ ID NO: 14. In some embodiments, the nucleic acid comprises or consists of the nucleotide sequence of SEQ ID NO: 15. In some embodiments, the nucleic acid comprises or consists of the nucleotide sequence of SEQ ID NO: 16. In some embodiments, the nucleic acid comprises or consists of a nucleotide sequence having between about 50%-99.5%, optionally at least about 50%, at least about 55%, at least about 60%, at least about 65%, at least about, at least about 70%, at least about 75%, at least about 80%, at least about 85%, at least about 90%, at least about at least about 95%, sequence identity to SEQ ID NO: 14, SEQ ID NO: 15, or SEQ ID NO: 16.

In some embodiments, the nucleic acid is an isolated nucleic acid.

In some embodiments, the nucleic acid encodes a plurality of the recombinant carbonic anhydrase polypeptides described herein. In some embodiments, the nucleic acid encodes a plurality of the recombinant carbonic anhydrase polypeptides comprising amino acid sequences of one or more of SEQ ID NOs: 1-11. In some embodiments, the nucleic acid encodes between 1-3 recombinant carbonic anhydrase polypeptides described herein. In some embodiments, the nucleic acid encodes between 1-3 the recombinant carbonic anhydrase polypeptides comprising amino acid sequences of one or more of SEQ ID NOs: 1-11.

Another aspect of the disclosures includes a vector or expression cassette encoding a recombinant carbonic anhydrase polypeptide described herein or the isolated nucleic acid described herein. The expression cassette or vector can be any suitable expression system. In some embodiments, the vector can be of yeast, fungi, insect, or mammalian origin. In some embodiments, the vector is a pET series vector. In some embodiments, the vector is a pET 21, pET22, pET28 or pET32 vector. In some embodiments, the vector is a pET-28(a) expression vector.

An aspect of the disclosure includes a cell comprising the vector or expression cassette described herein. In some embodiments, the cell is a bacteria cell, a yeast cell, a plant cell, an insect cell or a mammalian cell. In some embodiments, the cell is an isolated cell. In some embodiments, the cell is an E. coli cell. In some embodiments, the cell expresses a recombinant carbonic anhydrase polypeptide described herein. In some embodiments, the cell is an isolated cell.

Another aspect of the disclosure includes a composition comprising a recombinant carbonic anhydrase polypeptide described herein, an isolated nucleic acid described herein, a vector or expression cassette described herein, and/or a cell described herein, and optionally a diluent or carrier. Examples of diluents include 50 mM Tris (pH 8) and/or 200 mM sodium chloride. In some embodiments, the composition further comprises a protease inhibitor e.g., Aprotinin, Leupeptin, Pepstatin A and/or Phenylmethylsulfonyl fluoride (PMSF).

In some embodiments, the composition comprises a plurality of recombinant carbonic anhydrase polypeptides described herein. In some embodiments, the composition comprises a plurality of recombinant carbonic anhydrase polypeptides described herein selected from SEQ ID NOs: 1-11.

In some embodiments, the composition is a lyophilized composition. In some embodiments, the composition is a reconstituted lyophilized composition. In some embodiments, the composition is reconstituted in e.g., a suitable carrier or diluent. In some embodiments, the carrier or diluent is 50 mM Tris (pH 8.0) or 200 mM NaCl.

An aspect of the disclosure is a method of making a recombinant carbonic anhydrase polypeptide described herein, the method comprising culturing a host cell under conditions enabling the expression of the recombinant, carbonic anhydrase polypeptide, and recovering the recombinant carbonic anhydrase polypeptide.

In some embodiments, the method of making a recombinant carbonic anhydrase polypeptide described herein comprises: transforming a host cell with a vector encoding a recombinant carbonic anhydrase polypeptide described herein; culturing said transformed host cell under conditions whereby said recombinant carbonic anhydrase polypeptide is produced by said host cell; and recovering said recombinant carbonic anhydrase polypeptide from said host cells.

In some embodiments, the host cell is the cell described herein. In some embodiments, the method comprises one or more steps used in the Examples.

An aspect of the disclosure includes a use of the above mentioned recombinant carbonic anhydrase polypeptides, the nucleic acid defined herein, the vector or expression cassette described herein, the cell described herein, and/or the composition described herein, in an industrial process for capturing CO₂ from a CO₂-containing effluent or gas.

An aspect of the disclosure includes a recombinant polypeptide described herein, the nucleic acid defined herein, the vector or expression cassette described herein, the cell described herein, and/or the composition described herein, for use in capturing CO₂, optionally converting CO₂ into bicarbonate and H+ ions.

An aspect of the disclosure includes use of a recombinant polypeptide described herein, the nucleic acid defined herein, the vector or expression cassette described herein, the cell described herein, and/or the composition described herein, for capturing CO₂, optionally converting CO₂ into bicarbonate and H+ ions.

In some embodiments the recombinant polypeptide described herein, the nucleic acid defined herein, the vector or expression cassette described herein, the cell described herein, and/or the composition described herein, is for use as a biocatalyst for capture and/or sequestration of atmospheric CO₂. In some embodiments the recombinant polypeptide described herein is for use capturing and/or sequestering CO₂ from flue gas in aqueous solvents.

In some embodiments, the use comprises capturing CO₂ from a CO₂-containing effluent or gas.

An aspect described herein is a method for absorbing CO₂ from a CO₂-containing effluent or gas, the process comprising: contacting the CO₂-containing effluent or gas with an aqueous absorption solution to dissolve the CO₂ into the aqueous absorption solution; and providing the recombinant carbonic anhydrase polypeptide defined, the nucleic acid defined herein, the vector or expression cassette described herein, the cell described herein, and/or the composition described herein to catalyze the hydration reaction of the dissolved CO₂ into bicarbonate and hydrogen ions or the reverse reaction.

In some embodiments, the recombinant carbonic anhydrase polypeptides described herein are used in combination with an absorption solution comprising at least one absorption compound that aids in the absorption of CO₂. In some embodiments, the absorption solutions described herein may comprise at least one absorption compound such as: (a) a primary amine, a secondary amine, a tertiary amine, a primary alkanolamine, a secondary alkanolamine, a tertiary alkanolamine, a primary amino acid, a secondary amino acid, a tertiary amino acid, dialkylether of polyalkylene glycols, dialkylether or dimethylether of polyethylene glycol, amino acid or a derivative thereof, monoethanolamine (MEA), 2-amino-2-methyl-1-propanol (AMP), 2-(2-aminoethylamino)ethanol (AEE), 2-amino-2-hydroxymethyl-1,3-propanediol (Tris orAHPD), N-methyldiethanolamine (MDEA), dimethylmonoethanolamine (DMMEA), diethylmonoethanolamine (DEMEA), triisopropanolamine (TIPA), triethanolamine (TEA), diethanolamine (DEA), diisopropylamine (DTPA), methylmonoethanolamine (MMEA), tertiarybutylaminoethoxy ethanol (TBEE), N-2-hydroxyethyl-piperzine 2-amino-2-hydroxymethyl-1,3-propanediol (AHPD), hindered diamine (HDA), bi-(tertiarybutylaminoethoxy)-ethane (BTEE), ethoxyethoethanol-tertiarybutylamine (EEETB), bis-(tertiarybutylaminoethyl)ether, 1,2-bis-(tertiarybutylaminoethoxy)ethane and/or bis-(2-isopropylaminopropyl)ether, or a combination thereof; (b) a primary amine, a secondary amine, a tertiary amine, a primary alkanolamine, a secondary alkanolamine, a tertiary alkanolamine, a primary amino acid, a secondary amino acid, a tertiary amino acid; or a combination thereof; (c) dialkylether of polyalkylene glycols, dialkylether or dimethylether of polyethylene glycol, amino acid or derivative thereof, or a combination thereof; (d) piperazine or derivative thereof, preferably substituted by at least one of alkanol group; (e) monoethanolamine (MEA), 2-amino-2-methyl-1-propanol (AMP), 2-(2-aminoethylamino)ethanol (AEE), 2-amino-2-hydroxymethyl-1,3-propanediol (Tris or AHPD), N-methyldiethanolamine (MDEA), dimethylmonoethanolamine (DMMEA), diethylmonoethanolamine (DEMEA), triisopropanolamine (TIPA), triethanolamine (TEA), diethanolamine (DEA), diisopropylamine (DIPA), methylmonoethanolamine (MMEA), tertiarybutylaminoethoxy ethanol (TBEE), N-2-hydroxyethyl-piperzine (HEP), 2-amino-2-hydroxymethyl-1,3-propanediol (AHPD), hindered diamine (HDA), bis-(tertiarybutylaminoethoxy)-ethane (BTEE), ethoxyethoxyethanol-tertiarybutylamine (EEETB), bis-(tertiarybutylaminoethyl)ether, 1,2-bis-(tertiarybutylaminoethoxy)ethane and/or bis-(2-isopropylaminopropyl)ether; (f) an amino acid or derivative thereof, which is preferably a glycine, proline, arginine, histidine, lysine, aspartic acid, glutamic acid, methionine, serine, threonine, glutamine, cysteine, asparagine, valine, leucine, isoleucine, alanine, tyrosine, tryptophan, phenylalanine, taurine, N-cyclohexyl 1,3-propanediamine, N-secondary butyl glycine, N-methyl N-secondary butyl glycine, diethylglycine, dimethylglycine, sarcosine, methyl taurine, methyl-α-aminopropionicacid, N-(β-ethoxy)taurine, N-(β-aminoethyl)taurine, N-methyl alanine, 6-aminohexanoic acid, potassium or sodium salt of the amino acid, or any combination thereof; (g) a carbonate compound; (h) sodium carbonate, potassium carbonate, or MDEA; (i) sodium carbonate; or (j) potassium carbonate.

In some embodiments, the CO₂, capture processes described herein may comprise exposing the recombinant carbonic anhydrase polypeptides described herein to a temperature of up to 90° C., 91° C., 92° C., 93° C., 94° C., 95° C., 96° C., 97° C., 98° C., 99° C., 100° C., 101° C., 102° C., 103° C., 104° C., 105° C., or 106° C. In some embodiments, the CO₂, capture processes described herein may comprise exposing the recombinant carbonic anhydrase polypeptides described herein to a temperature of up to about 104° C. or 106° C. In some embodiments, the CO₂, capture processes described herein may comprise exposing the recombinant carbonic anhydrase polypeptides described herein to a temperature of about or up to about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., or 106° C.

In some embodiments, CO₂ capture processes described herein may comprise one or more additional features (e.g., relating to overall CO₂ capture system, absorption unit, desorption unit, separation unit, measurement device, and/or process parameters/conditions) as described in WO/2016/029316 and/or WO/2017/035667, and/or Alvizo, Oscar et al. “Directed evolution of an ultrastable carbonic anhydrase for highly efficient carbon capture from flue gas.” Proceedings of the National Academy of Sciences of the United States of America vol. 111,46 (2014): 16436-41.

An aspect of the disclosures includes a method for removing carbon dioxide from a gas stream comprising the step of contacting the gas stream with a composition comprising a recombinant carbonic anhydrase polypeptide described herein, the nucleic acid defined herein, the vector or expression cassette described herein, the cell described herein, and/or the composition described herein, whereby carbon dioxide from the gas stream is dissolved in the solution and converted to hydrated carbon dioxide.

In some embodiments, the method further comprises the step of isolating the solution comprising hydrated carbon dioxide and contacting the isolated solution with hydrogen ions and a recombinant carbonic anhydrase polypeptide described herein, thereby converting the hydrated carbon dioxide to carbon dioxide gas and water.

In some embodiments the present disclosure provides methods for removing (e.g., extracting and sequestering) carbon dioxide from a gas stream comprising the step of contacting the gas stream with a solution comprising a recombinant carbonic anhydrase polypeptide of a recombinant carbonic anhydrase polypeptide of the disclosure having an improved property (e.g., increased activity and/or thermostability), whereby carbon dioxide is removed from the gas stream by dissolving into the solution where it is converted to hydrated carbon dioxide by the carbonic anhydrase polypeptide. In another embodiment, the method can comprise the further step of isolating the solution comprising the hydrated carbon dioxide and contacting the isolated solution with hydrogen ions and a recombinant carbonic anhydrase polypeptide, thereby converting the hydrated carbon dioxide to carbon dioxide gas and water. Thus, it is contemplated that the solution can be removed from contact with the gas stream (e.g., isolated after some desired level of hydrated carbon dioxide is reached) and further treated with a carbonic anhydrase polypeptide described herein to convert the bicarbonate in solution into carbon dioxide gas, which is then released from the solution and captured e.g., into a pressurized chamber.

In some embodiments, the methods for removing (e.g., extracting and sequestering) carbon dioxide from a gas stream disclosed herein can be used in processes for removing carbon dioxide from the flue gas produced by a fossil fuel (e.g., coal-fired) power plant. Equipment and processes that can employ the recombinant carbonic anhydrases in processes to remove carbon dioxide from the flue gas of fossil fuel power plants have been described—see e.g., U.S. Pat. No. 6,143,556, US patent publication no. 2007/0004023A1, and PCT publications WO98/55210A1, WO2004/056455A1, and WO2004/028667A1, and/or Alvizo, Oscar et al. “Directed evolution of an ultrastable carbonic anhydrase for highly efficient carbon capture from flue gas.” Proceedings of the National Academy of Sciences of the United States of America vol. 111,46 (2014): 16436-41, each of which is hereby incorporated by reference herein. An aspect of the disclosure includes a recombinant carbonic anhydrase polypeptide described herein, the nucleic acid defined herein, the vector or expression cassette described herein, the cell described herein, and/or the composition described herein, for use in accelerating the weathering of minerals e.g., limestone or for enhancing carbon storage or sequestration in a water source, e.g., an ocean or sea.

An aspect of the disclosure includes a method of accelerating weathering of minerals e.g., limestone, or for enhancing carbon storage or sequestration in a water source, e.g., an ocean or sea, the method comprising contacting a water source, optionally a mineral rich water source, optionally an ocean or sea, with a recombinant carbonic anhydrase polypeptide described herein, the nucleic acid defined herein, the vector or expression cassette described herein, the cell described herein, and/or the composition described herein, optionally such that the recombinant carbonic anhydrase polypeptide catalyzes the reversible hydration of CO₂ to bicarbonate, thereby increasing the availability of reactive carbonate species that can combine with calcium or magnesium ions in oceans or mineral-rich environments to form stable carbonates. For example, Carbonic anhydrase can significantly enhance the process of accelerated mineral weathering for carbon capture and storage (CCS). By rapidly catalyzing the reversible hydration of CO₂ to bicarbonate, CA increases the availability of reactive carbonate species that can combine with calcium or magnesium ions in oceans or mineral-rich environments to form stable carbonates. This biocatalytic acceleration reduces the kinetic barriers of natural weathering, enabling faster and more efficient CO₂ sequestration in both geological formations and industrial applications. Incorporating carbonic anhydrase into mineralization strategies, therefore, offers a sustainable, low-energy pathway to enhance long-term carbon storage. Such method are described e.g., in Vibbert Hunter & Park, 2022, “Harvesting, storing, and converting carbon from the ocean to create a new carbon economy: Challenges and opportunities”, Frontiers in Energy Research (10); Antonopoulou, et al., “Accelerated carbonate weathering by immobilized recombinant carbonic anhydrase.” Carbondioxide-removal.eu, 9 Mar. 2025; and Subhas, Adam V., et al. “Catalysis and chemical mechanisms of calcite dissolution in seawater.” Proceedings of the National Academy of Sciences, vol. 114, no. 31, 2017, pp. 8175-8180. An aspect of the disclosure includes a method of identifying recombinant carbonic anhydrase polypeptides, optionally with improved thermostability and/or activity, e.g., hydratase and/or esterase activity using ancestral sequence reconstruction methods described e.g., in the Examples.

An aspect of the disclosures includes a kit comprising one or more recombinant carbonic anhydrase polypeptides described herein, an isolated nucleic acid described herein, the vector or expression cassette described herein, the host cell described herein, and/or a composition described herein, and optionally instructions for use. In some embodiments, the kit further comprises one or more vial or tube. In some embodiments, the kit is for use in one or more of the methods and/or uses described herein. In some embodiments, the kit further comprises a protease inhibitor and/or lysis buffer.

In some embodiments, the kit is for use in the methods or uses described herein.

Uses of the methods described herein are also encompassed.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, in certain methods described herein that include more than one step or act, the order of the steps or acts of the method is not necessarily limited to the order in which the steps or acts of the method are recited unless the context indicates otherwise.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### EXAMPLES

### Example 1

Anthropogenic atmospheric CO₂ emissions from industries that rely on fossil fuels are the major contributors to climate change and global warming worldwide. According to the annual Global Carbon Project Report (2023), there is an estimated 1.1% increase in global fossil CO₂ emissions relative to 2022. Due to this rapid increase in the level of atmospheric CO₂, efforts to reduce its release into the atmosphere have been intensified, particularly by implementing carbon capture and sequestration (CCS) technologies within the industries related to higher CO₂ emission. Presently, the widely reported, practical and effective CCS technology involves post-combustion CO₂ capture using amine solvents [1] to separate CO₂ from flue gas released from industries. However, the use of amine requires substantial energy for CO₂ release and solvent regeneration, resulting in high operational costs and sub-optimal CO₂ absorption kinetics. To address these limitations, biomimetic or enzymatic approaches using carbonic anhydrase (CA) enzymes present a compelling alternative for CO₂ capture, offering economical, selective, and environment-friendly solution.

CA catalyzes the reversible hydration of CO₂ to bicarbonate and protons. Renowned for its remarkable kinetics and substrate turnover rate reaching up to a million CO₂ molecules per second, CA stands out as an exceptional biocatalyst for efficient capture of atmospheric CO₂. The use of CA in capturing CO₂ from flue gas in aqueous solvents has already been established leading to its commercialization by industries for biomimetic CO₂ capture and storage.

Industrial application of CA requires the enzyme to be functional under high temperature and harsh conditions prevailing during post-combustion CO₂ capture processes. However, the key limitation in deploying natural CAs for these applications lies in their susceptibility to inactivation and denaturation at elevated temperature involved in the CO₂ desorption. Furthermore, the long-term operational stability, recovery, and recycling of the enzyme complicates the use of CAs in industrial settings. Therefore, enhancing the thermostability and reusability of CAs whilst preserving their catalytic activity is still a paramount objective. Application of a well-known method for stability enhancement, Ancestral sequence reconstruction (ASR), has been largely overlooked for CAs.

Herein, the ASR technique is applied to engineer an α-family CA, focusing on enhancing thermostability and preserving the activity of CAs for CO₂ capture applications. Ancestral forms of CA through were generated through a targeted process involving a carefully chosen set of extant CA enzymes. The recombinant carbonic anhydrases generated herein possess favourable properties such as extreme stability, improved activity with an ability to refold after thermal denaturation and still shows catalytic activity after refolding. Consequently, this study not only addresses the protein engineering gap in obtaining stable CAs, but also provides compelling evidence for enhanced thermostability of the recombinant carbonic anhydrases generated herein compared to the extant enzyme. This disclosure contributes to advancing the application of CAs in industrial CO₂ capture applications.

### Results and Discussion

### Ancestral Sequence Reconstruction

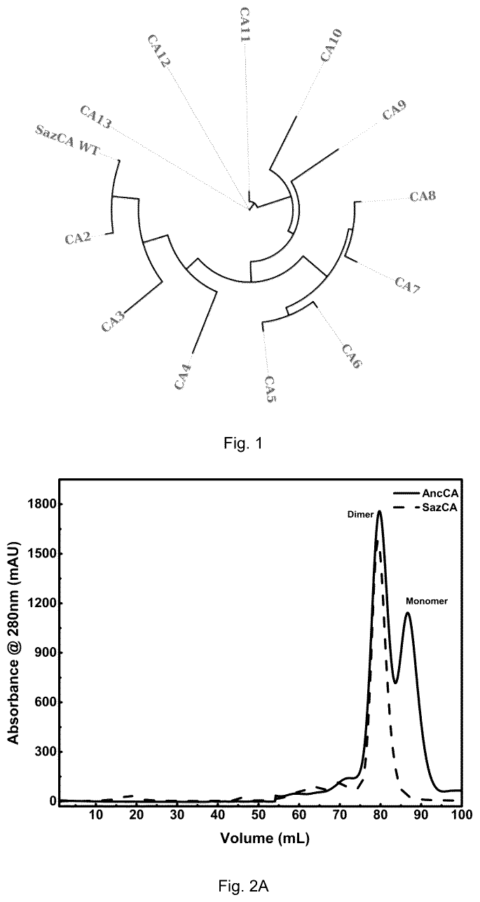

To obtain a highly stable ancestral form of CA, we shifted our focus towards Sulphurihydrogenibium azorense (SazCA), an enzyme from α-family CAs. We performed a homology search using the SazCA sequence as a query and obtained its closest homologs. Given the low sequence similarity among the members of the α-family CAs, an initial BLAST search using SazCA yielded only 100 sequence hits. Following sequence filtering as described in the methods section, a total of 13 sequences remained, which were then aligned using the Multiple Alignment using Fast Fourier Transform (MAFFT) program. The sequence alignment was further curated to eliminate sequence gaps, and a phylogenetic tree was constructed. The best-fitting evolutionary model was identified using ModelFinder [4], implemented in the IQ-TREE program (FIG. 1). For the given alignment, WAG+I+G4 was identified as the best-fitting model based on the Bayesian Information Criterion (BIC). The final log-likelihood of the tree was −3189.309. Resampling of the tree with the bootstrap of 1000 was done simultaneously by the program and the ancestral amino acids for each position of the alignment were reconstructed with the same evolutionary model as above (WAG+I+G4) utilizing the maximum likelihood reconstruction method available in IQTree. The, had an Amino acid sequence comparison of ancestral Node7 (AncCA) (FIG. 1) with the extant enzymes showed 86% and 69.9% sequence identity to SazCA and SspCA, respectively. To further investigate the stability and enzymatic activity of AncCA, genes encoding the inferred amino acid sequence for AncCA, and extant SazCA were synthesized and expressed in E. coli using a Genscript pET-28(a) expression vector system. Both enzymes were successfully expressed in soluble form and readily purified using affinity and size exclusion chromatography.

### Oligomeric State of AncCA

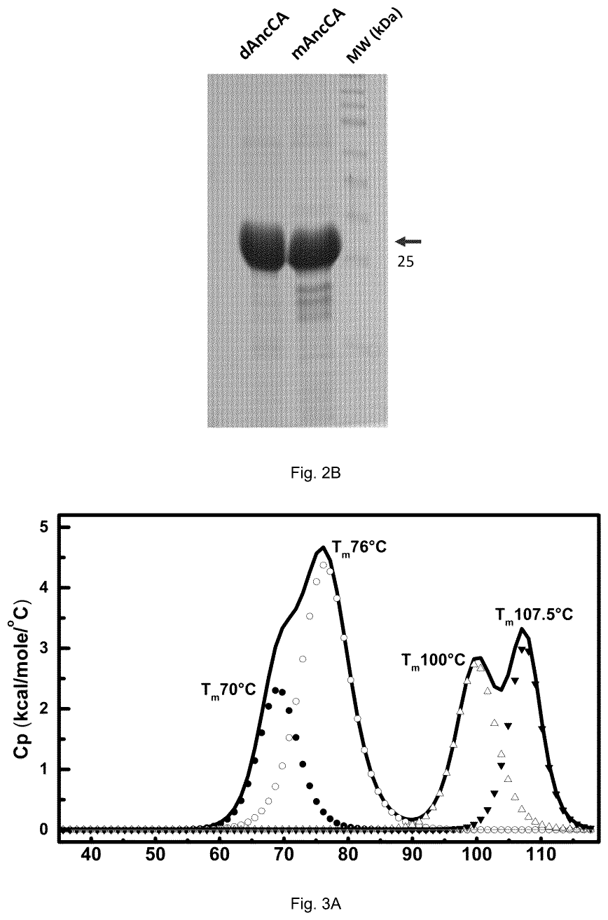

Using size exclusion chromatography (SEC) the oligomeric state of AncCA was confirmed. The elution profiles in FIG. 2A show that AncCA exists in two different oligomeric states, corresponding to the molecular weight of a dimer (dAncCA) (elution volume ˜10.5 mL) and a monomer (mAncCA) (elution volume ˜12 mL). In contrast, SazCA predominantly migrates as a single molecular species with the retention volume corresponding to the molecular weight of a dimer. To assess the purity, both dAncCA and mAncCA were eluted as separate fractions and were analyzed on SDS-PAGE gel and were shown to be 95% pure (FIG. 2B). In addition to the high expression for AncCAs, it had a resistance to aggregation even after heating at 100° C., as opposed to SazCA, which showed complete visible aggregation at high temperatures, indicating the higher thermostability of both AncCA enzymes (dAncCA and mAncCA) compared to SazCA.

### Thermal Stability

### Differential Scanning Calorimetry (DSC)

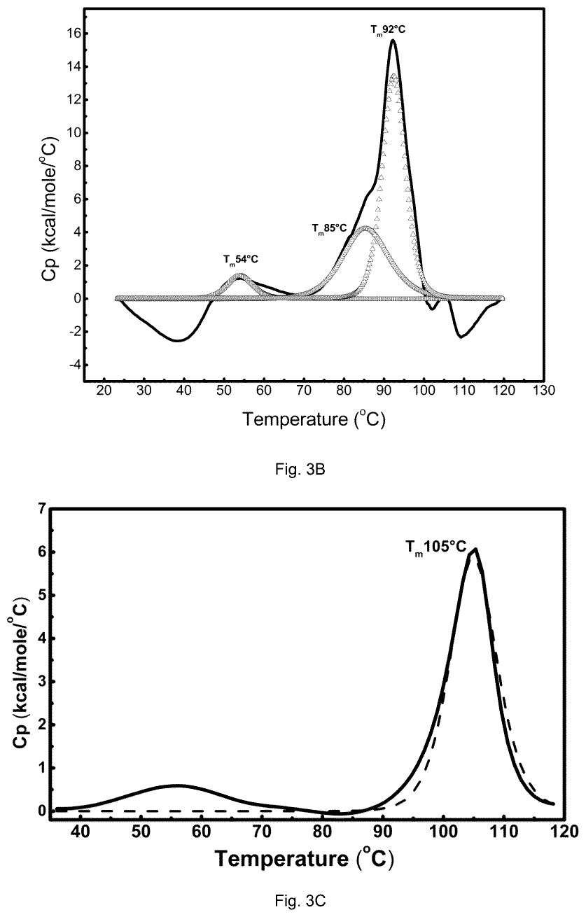

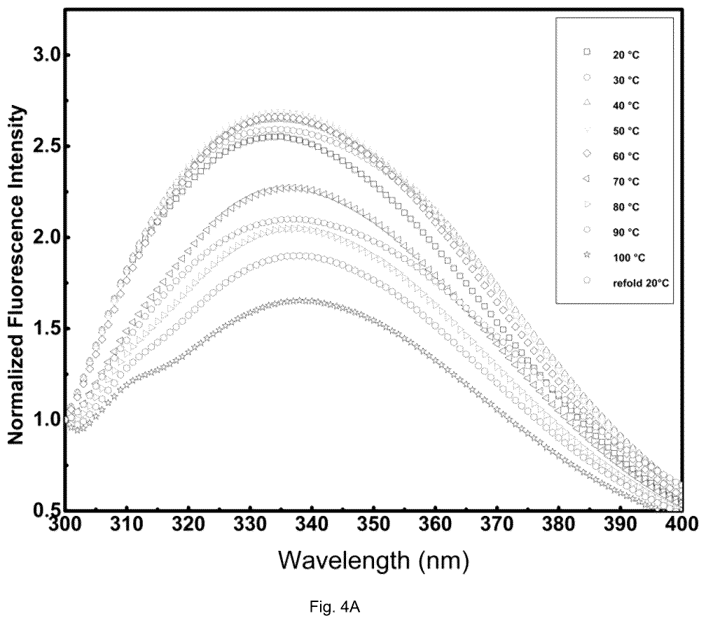

To test and evaluate the thermostability of AncCA, thermal unfolding experiments were performed using differential scanning calorimetry (DSC) and compared to SazCA. FIG. 3A, C, show DSC scans measured for AncCA and SazCA respectively, and the calculated Tm and ΔH values are listed in Table 1. The thermal unfolding for both enzymes were complex and do not follow two-state unfolding process. For AncCA (FIG. 3A), unfolding transitions were observed at temperature around ˜69° C., ˜78° C., ˜99° C. and a final unfolding peak at ˜107° C. Similarly, DSC traces for SazCA (FIG. 3C), also displayed multiple transitions at temperatures around ˜52° C., ˜84° C. and ˜92.5° C. The small peak at ˜52° C. represents the structural changes in the enzyme, consistent with prior reports for SazCA. The transition at ˜84° C. from temperature-induced conformational fluctuations in the enzyme, culminated to the final unfolding at ˜92.5° C. Comparing the Tm for final unfolding transitions suggests that AncCA undergoes complete denaturation at higher temperature of ˜107° C. compared to SazCA. AncCA exhibits an approximately 15° C. increase in its temperature for unfolding than SazCA. Multi-step unfolding transitions results from the existence of intermediate states due to structural/conformational changes within the enzyme across the range of temperatures. As the data were fitted to a non-two-state model, the different peaks could represent intermediate states for each enzyme, leading to its final denaturation at high temperatures. To evaluate the thermodynamic cooperativity of unfolding for AncCA, the ratio for calorimetric enthalpy (ΔHcal) to Van′t Hoff enthalpy (ΔHvh) were determined for each transition (Table 1). Data reveals that for all the transitions for AncCA, the ratio was less than 1 suggesting that the unfolding process was thermodynamically non-cooperative. A subsequent reversible scan (scan2) following the denaturation and cooling revealed that SazCA undergoes irreversible denaturation (data not shown), accompanied by enzyme aggregation and no recovery of the enzyme activity. On the contrary, for AncCA (FIG. 3B), reversible temperature-induced transitions resulted in a distinct profile compared to scan1 (FIG. 3A). Interestingly, the reversible thermal unfolding for AncCA resulted in overlapping transition only at higher temperatures (TM ˜105° C.) (FIG. 3B). The presence of a single transition peak and elimination of all the other intermediate states in scan2 led to the conclusion that the AncCA renatured to highly thermally stable conformation.

TABLE 1
Thermodynamic parameters for protein unfolding.

| | T_{M} | ΔH_{Cal} | ΔHᵥₕ | ΔH_{Cal}/ |
|---|---|---|---|---|
| Enzymes | (° C.) | (kcal/mol) | (kcal/mol) | ΔHᵥₕ |
| SazCA | 77 | 1.49 | 0.65 | 2.3 |
| | 92.5 | 1.55 | 1.24 | 1.25 |
| dAncCA | 69 | 0.19 | 1.14 | 0.16 |
| | 76.5 | 4.41 | 9.65 | 0.45 |
| | 100 | 0.22 | 1.35 | 0.16 |
| | 107.5 | 0.2 | 1.74 | 0.11 |
| dAncCA | 105 | 0.6 | 1.10 | 0.54 |
| (Scan2) | | | | |

To gain further insights into the thermal unfolding process, activation energy (EA) parameters were determined for individual transition peaks in the DSC. Rates for each unfolding transition were obtained and Arrhenius plots were generated for both AncCA (FIG. 3D) and SazCA (FIG. 3E). The data spanned a wide temperature range, so the linear fits were approximated by excluding the temperatures on either side to capture the overall trend. For both enzymes, however, there was a clear deviation from linearity when data was plotted considering the full temperature range. The activation energy for each peak was deduced from the slope of Arrhenius plot. For both SazCA and AncCA, EA for the first unfolding transition was lower than the second and third transition peaks. The unfolding rate and EA show an increasing trend with rising temperature, with the final peak at Tm ˜107° C. showing the highest EA for AncCA. Specifically, the EA for the final unfolding peaks were 757 KJ mol-1 and 990 KJ mol-1 for SazCA and AncCA, respectively. The higher EA for AncCA compared to SazCA indicates that the rate of denaturation for the former is slower as it must overcome a larger energy barrier to unfold, suggesting greater stability for AncCA. The DSC scan for the refolding transition of AncCA in scan 2 shows an EA of 715 KJ mol-1 for a single peak transition. Thus, findings from the DSC experiments clearly demonstrate that (i) the ancestral enzyme, AncCA, exhibits greater thermostability compared to extant enzyme, SazCA (ii) has tendency to renature after denaturation and (iii) Higher EA for denaturation of AncCA suggests slower denaturation hence more stability of the enzyme.

### Fluorescence Spectroscopy

Given the complexity of the thermal unfolding process for AncCA, as revealed by DSC, this process was further investigated using intrinsic tryptophan (Trp) fluorescence spectroscopy. This method allows analysis of whether temperature changes alter the conformation around the active site, as three of four Trp residues in the enzyme are located within the active site pocket. Fluorescence experiments were conducted on both dimer (dAncCA) and monomer (mAncCA) fractions of the enzyme and the changes in the fluorescence were monitored at different temperatures.

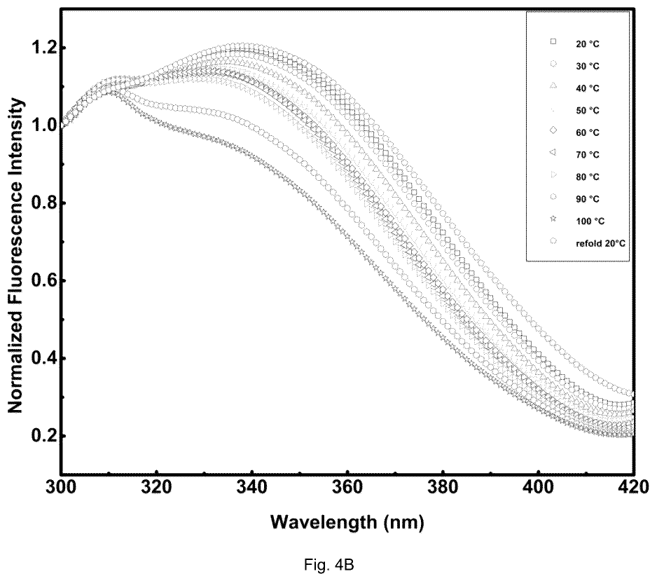

The fluorescence spectra for dAncCA (FIG. 4A) shows that the fluorescence signal (maxima @ 333 nm) remained relatively unchanged between 20° C. to 60° C. and were characterized by sharp and symmetrical peaks. However, a sudden decrease in fluorescence intensity above 60° C. was observed, accompanied by a slight shift in the spectrum for 70° C. to 100° C. This observation indicates that the enzyme undergoes conformational transition above 60° C., leading to the exposure of buried Trp residues to the solvent. These results also corelates with the DSC transition observed at 70° C.-80° C., confirming the conformational fluctuation in the active site of the enzyme around this temperature. To confirm the refolding of the enzyme after heat denaturation, we performed fluorescent intensity measurements on the enzyme cooled back to 20° C. after heating to 100° C. The data reveal that the renatured enzyme regains fluorescence intensity close to the fluorescence peak observed at 80° C., suggesting that the refolded enzyme assumes a conformation different from the native enzyme, very similar to the one attained after heating at 80° C.

For mAncCA (FIG. 4B), the intrinsic fluorescence spectrum differed from that of the dAncCA, with fluorescence maxima at 337 nm, reflecting the solvation of aromatic residues involved in the dimer-dimer interface. No significant changes were observed in the fluorescence signal until 80° C. However, there was a decrease in intensity at 90° C. mAncCA regained all its fluorescence intensity after the denatured enzyme was cooled back to 20° C., suggesting that the conformation of renatured enzyme was similar to its native form. The EA of thermal unfolding calculated from the Arrhenius plots (FIG. 4C), reveals that dAncCA (75.2 KJ/mol) has a higher Ea than mAncCA (44.6 KJ/mol). The slower unfolding for dAncCA could be the reason for its failure to completely renature, whereas the fast-unfolding process for mAncCA might allow the enzyme to quickly fold back to its native state.

### Circular Dichroism (CD)

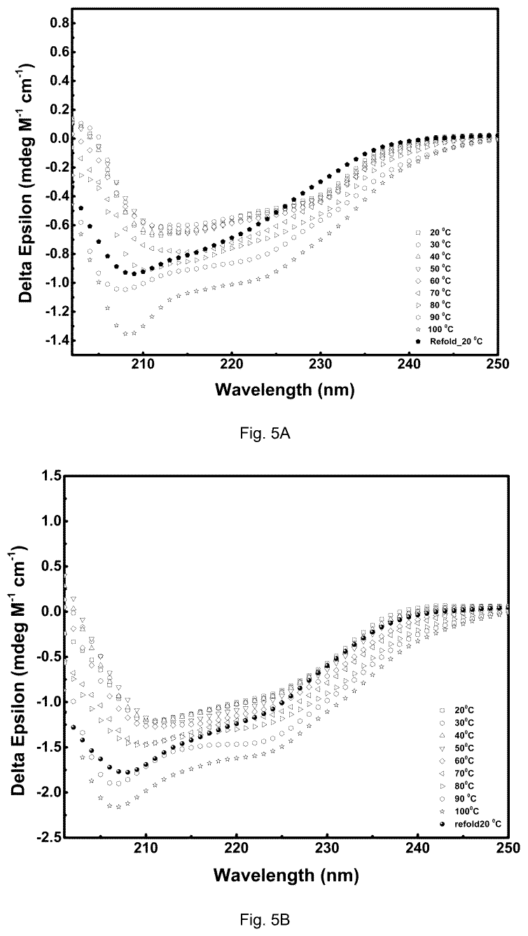

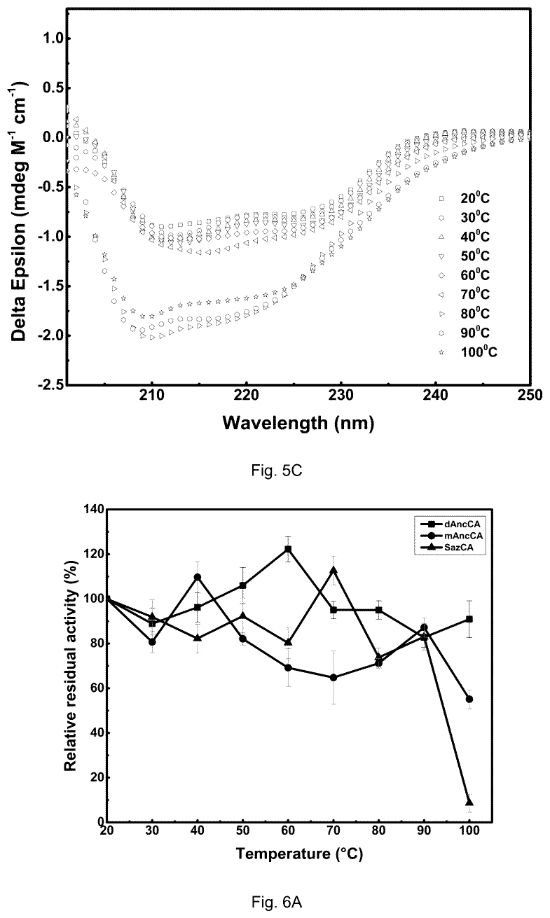

To analyse the effect of increasing temperature on the conformational changes in secondary structure of the enzymes, thermal unfolding experiments were conducted and the secondary structure changes were monitored using circular dichroism (CD) spectroscopy. The far-UV CD spectra obtained for dAncCA, mAncCA and SazCA, from 20° C. to 100° C. and cooled back to 20° C., are shown in FIG. 5 A-C. The spectra for dAncCA (FIG. 5A), obtained between temperatures 20° C. to 60° C. were almost similar, suggesting no significant changes in the secondary structural contents. However, above 70° C., a shift in the spectra was observed that persisted at 80° C., 90° C. and 100° C., indicating alterations in the secondary structure content at higher temperatures. These findings were consistent with the fluorescence experiments (FIG. 4A), which showed a decrease in fluorescence signal after 60° C., confirming that the decrease was due to changes in the secondary structure content of the enzyme. Further, the CD spectra of the cooled sample (i.e., cooled back to 20° C. after heating to 100° C.) revealed that the spectra differed from the native structure. The spectra was very similar to those obtained at higher temperatures. This observation is also supported by the DSC scan2 (after first unfolding), where the renatured enzyme's unfolding transition was only observed at higher temperatures, suggesting that the renatured enzyme adopts a conformation very similar to the structure attained at higher temperatures.

The percentage secondary structure content at each temperature was determined using BeStSel and is presented in Table 2. At 20° C., dAncCA consists of 3.6% α-helix, 41% anti-parallel β-sheet, 17.2% β-turn, and 38.1% random coil content. However, from 80° C. to 100° C., the enzyme shows an increase in α-helices (6.3%) and reduction in β-sheet (31.4%) content. After renaturation, the enzyme attained structure similar to the 60° C. heated sample, but with the larger random coil content. These results emphasize that the enzyme undergoes a conformational transition at high temperatures and this conformation adopted after renaturation contributes to the stability of the enzyme.

TABLE 2
Analysis of (%) secondary structure content for CD spectrum
of dAncCA, mAncCA and SazCA at various temperatures.

| | | 20° | 30° | 40° | 50° | 60° | 70° | 80° | 90° | 100° | |
|---|---|---|---|---|---|---|---|---|---|---|---|
| | Structures | C. | C. | C. | C. | C. | C. | C. | C. | C. | Cooled |
| | α-helix | 3.6 | 2.1 | 1.0 | 3.6 | 3.9 | 2.2 | 5.9 | 5.9 | 6.3 | 3.9 |
| | β-sheet | 41 | 38.2 | 40 | 38.4 | 39.9 | 39.3 | 39.6 | 24.3 | 31.4 | 31.5 |
| dAncCA | β -turn | 17.2 | 19.0 | 19.5 | 15.2 | 15.9 | 14.4 | 17.5 | 13.6 | 18.3 | 15.6 |
| | Random coil | 38.1 | 40.7 | 39.4 | 42.9 | 40.3 | 44.1 | 37 | 56.2 | 43.9 | 49 |
| | α-helix | 3.2 | 5.1 | 9.1 | 8.3 | 5.9 | 4.3 | 8.1 | 5.1 | 7.9 | 4.2 |
| mAncCA | β-sheet | 27.2 | 35.4 | 20.4 | 25.2 | 23.1 | 22.8 | 22.6 | 10.6 | 14.2 | 25 |
| | parallel β-sheet | 0 | 0 | 0 | 1.5 | 0 | 4.7 | 5.7 | 3.9 | 9.1 | 0 |
| | β -turn | 17.7 | 19.9 | 14.4 | 12.2 | 15.9 | 18.6 | 14.8 | 19.3 | 15.8 | 20.5 |
| | Random coil | 51.9 | 39.5 | 56.1 | 52.7 | 55.1 | 49.6 | 48.8 | 61.1 | 53.1 | 50.3 |
| | α-helix | 3 | 1.3 | 7.3 | 2.5 | 1.1 | 2.3 | 8.0 | 12.7 | 11.7 | 7.6 |
| SazCA | β-sheet | 29.5 | 30.2 | 36.4 | 39.3 | 44.5 | 35.6 | 34 | 17.4 | 26.9 | 23.3 |
| | β -turn | 19.1 | 15.5 | 15.2 | 15.3 | 19.7 | 21.8 | 20.3 | 31.5 | 18 | 18.6 |
| | Random coil | 48.4 | 53.0 | 41.1 | 42.4 | 34.7 | 40.3 | 37.7 | 38.4 | 42.9 | 50.4 |

Similarly, for mAncCA (FIG. 5B), the CD spectra were very similar for temperatures between 20° C. to 80° C. suggesting higher structural stability for mAncCA. Beyond this temperature, a change in the spectra was observed at 90° C. and 100° C. compared to the native spectra at 20° C. The BeStSel analysis suggests that the enzyme consists of 3.2% α-helix, 27.2% anti-parallel β-sheet, 17.7% β-turn, and 51.9% random coil content at 20° C. The native mAncCA contains less β-sheet content and a higher coiled structure content compared to dAncCA. At higher temperatures, mAncCA exhibits higher α-helix and lower anti-parallel β-sheet content. Interestingly, at high temperatures, mAncCA shows a significant amount of parallel β-sheet content, which was not observed in the dAncCA. The spectra and the percentage secondary structure content calculated for the renatured mAncCA shows that the enzyme refolds to a structure similar to the native enzyme. This was also confirmed from fluorescence experiments (FIG. 3b), where the fluorescence intensity of the renatured sample was regained after cooling the sample back after denaturation.

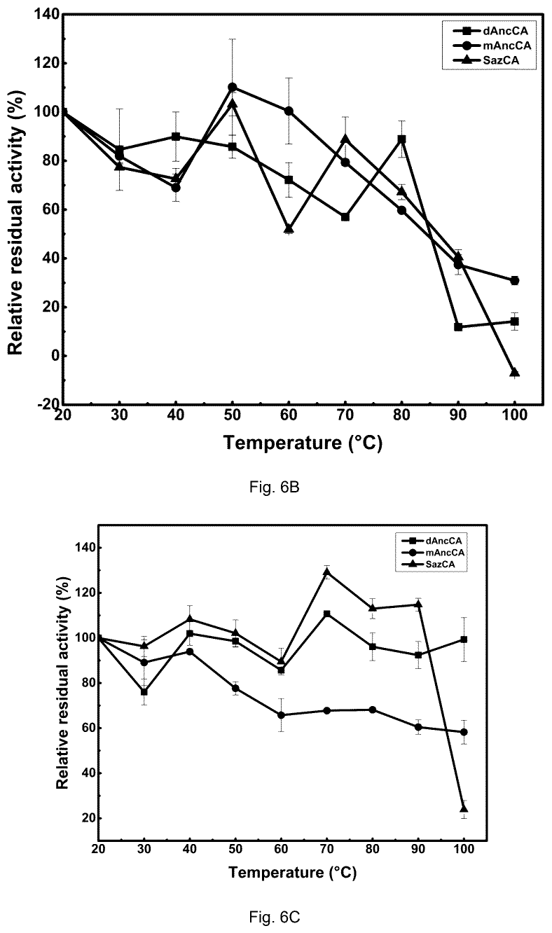

Overall, the CD secondary structure analysis for AncCAs shows a higher β-sheet content which agrees with the structures available for most of the characterized α-family CAs including SazCA. The structural content for SazCA is already known from its crystal structure but the effect of temperature on its secondary structure content has not yet been determined. Therefore, we also monitored CD spectra for the thermal unfolding of SazCA (FIG. 5C). The spectra for SazCA were similar for temperatures between 20° C.-70° C., however a significant spectral shift was observed at 80° C., 90° C. and 100° C., suggesting changes in the secondary structure at high temperatures. These findings are also consistent with the observed heat capacity increase at 80° C. in DSC. The calculated percentage secondary structure at 20° C. for SazCA revealed 3% α-helix, 29.5% anti-parallel β-sheet, 19% β-turn, and 48.4% random coil content. The enzyme shows increased α-helix content at 80° C., 90° C. and 100° C. Although SazCA retains some secondary structure at 100° C., it loses all enzymatic activity at this temperature (FIG. 6), suggesting that the conformation attained at 100° C. lacks a proper active site geometry. The scans obtained immediately after cooling to 20° C. indicate that the enzyme needs more time to refold back to the original conformation, unlike mAncCA, which refolded more quickly.

Comparing the CD-based secondary structure content analysis for native dAncCA, mAncCA and SazCA suggests: (i) at 20° C., all three enzymes show similar α-helix but differ in the β-sheet content with dAncCA containing more anti-parallel β-sheet content (41%) compared to mAncCA (27%) and SazCA (29.5%). (ii) For all 3 enzymes, an increase in α-helix and a decrease in β-sheet content at high temperatures were observed when compared to the native structure at 20° C. (iii) Secondary structure content for mAncCA differs from dAncCA and SazCA due to the presence of parallel β-sheet that was absent in the other two enzymes and (iv) lastly, comparing renatured dAncCA versus mAncCA shows less α-helix and more β-sheets for dAncCA, whereas mAncCA has more α-helix content and less β-sheet. FIG. 5D, shows changes in delta epsilon at 222 nm as a function of increasing temperature. It is observed that the structural changes are gradual for dAncCA and mAncCA, but for SazCA, there is a cooperative loss of native structure and a sudden transition to a more α-helix state above 70° C.

### Hydratase Activity

The enzyme activity to convert CO₂ into bicarbonate and H+ ions was measured using a pH-based assay that detects the color change upon proton release during the CO₂ hydration reaction. The specific activity measured for SazCA (accession number ACN99362) (32828 WAU/mg) agrees with the previously reported value [5]. dAnc_CA (61227 WAU/mg) exhibited a 1.9- and 2-fold higher specific activity compared to SazCA and mAncCA (30104 WAU/mg), respectively, rendering it exceptionally active. AncCA displays extreme thermostability and activity. The steady state kinetic parameters of hydratase activity for dAncCA, mAncCA and SazCA were also determined using CO₂ as a substrate. As reported in Table 3, dAncCA exhibited higher CO₂ hydration activity compared to mAncCA and SazCA. The maximum reaction rate (Vmax) for dAncCA was 4-fold times higher than that of mAncCA and 3-fold times higher than SazCA. Additionally, dAncCA displayed 3.1-fold higher catalytic efficiency than SazCA, suggesting an exceptional activity surpassing any known CAs.

TABLE 3
CO₂ Catalytic activities of dAncCA, mAncCA and SazCA.

| | Vₘₐₓ | K_{cat} | Kₘ | K_{cat}/Kₘ |
|---|---|---|---|---|
| | (M s⁻¹) | (s⁻¹) | (mM) | (M⁻¹ s⁻¹) |
| dAncCA | 0.012 | 4.3 × 10⁷ | 23.4 | 18.5 × 10⁸ |
| mAncCA | 0.003 | 1.5 × 10⁷ | 29.7 | 5.2 × 10⁸ |
| SazCA | 0.004 | 1.4 × 10⁷ | 25 | 5.5 × 10⁸ |

### Effect of Temperature on Hydratase Activity

Further, to understand the stability-activity relationship, enzyme activity assays were performed at different temperatures and time intervals to analyze its effect on the unfolding of the enzyme's active site.

Short-term heat treatment (30 min.): Each enzyme (dAncCA, mAncCA and SazCA) was heat-treated at different temperatures ranging from 20° C.-100° C. for 30 minutes and cooled on ice. The residual CO₂ hydration activities were then assessed and compared to the initial activity of the untreated enzyme at 20° C. The decrease in activity represents the fraction of enzyme that was inactivated after heating. As shown in FIG. 6A, dAncCA displayed almost similar residual activities at all temperatures except 60° C., where a 22% increase in residual activity was observed. A plausible interpretation for this observation relates to the conformational changes observed around this temperature in the DSC (FIG. 2A), and fluorescence (FIG. 3A) resulted in increased active site accessibility by the substrate (thermostimulation effect). Similar thermostimulation effects were also observed in mAncCA at 90° C. (consistent with the fluorescence results in FIG. 3B) and in SazCA at 70° C. (corresponding to the DSC transition peak in FIG. 2C). Moreover, dAncCA and mAncCA retained almost 91% and 55% of their activity at 100° C. compared to the untreated sample, whereas SazCA showed a significant loss of activity, retaining only 8% at 100° C.

Long heat treatment (180 min.): Following the short-term heat treatment, the impact of prolonged heat exposure on the enzymes by incubating them at various temperatures for 180 minutes and then assessing the residual activity for the CO₂ substrate. Comparing the relative residual activities of the enzymes (FIG. 6B), dAncCA retained maximum activity (89%) at 80° C. even after heating for a longer period, whereas mAncCA and SazCA shows 60% and 67% activity, respectively at the same temperature. After heating at 100° C., dAncCA and mAncCA were still active for CO₂ hydration, whereas SazCA was completely inactive at temperatures higher than 90° C., indicating a more pronounced loss of activity with prolonged heat exposure. It is interesting to observe that after heating for a longer time, mAncCA shows 17% higher activity than dAncCA at 100° C. implying that mAncCA is heat stable for longer durations and the prolonged heating has less effect on mAncCA than on dAncCA. These results collectively suggest that AncCA exhibits remarkable thermostability and maintains its activity more effectively over extended periods of heat exposure.

### Effect of Temperature on Esterase Activity

In addition to the CO₂ hydration activity, CAs have been known to possess promiscuous esterase activity, using β-nitrophenyl acetate (β-NPA) as a substrate. Therefore, the esterase activities of the enzymes at different temperatures ranging from 20-100° C. for 30 minutes were measured, and then compared the residual activities to the initial activity at 20° C. (FIG. 6C). Similar to hydratase activity, dAncCA retained nearly all of its esterase activity (99.7%) at 100° C., whereas mAncCA retained 76% of its activity. In contrast, SazCA was completely inactive at 100° C. for esterase activity. These results indicate that AncCAs were more thermoactive and thermostable at higher temperatures compared to the extant enzyme SazCA.

### Effect of Repeated Heating and Cooling Cycles on Oligomeric State and Activity

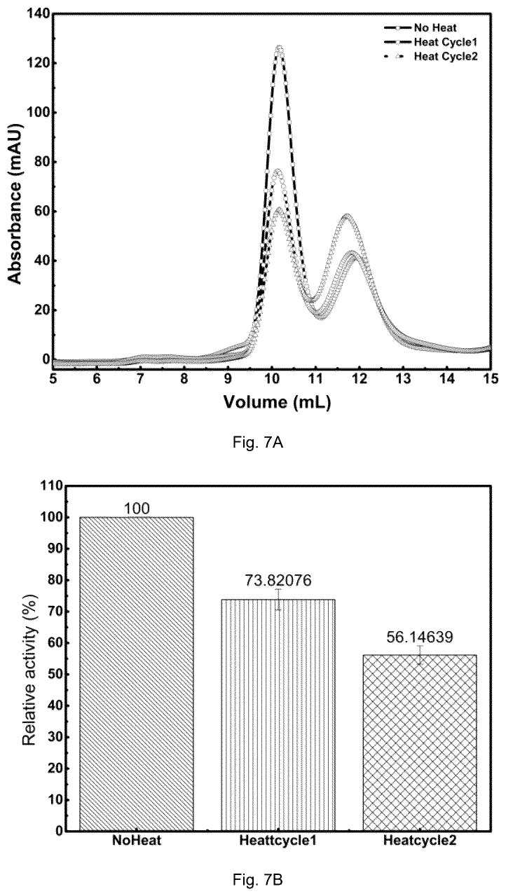

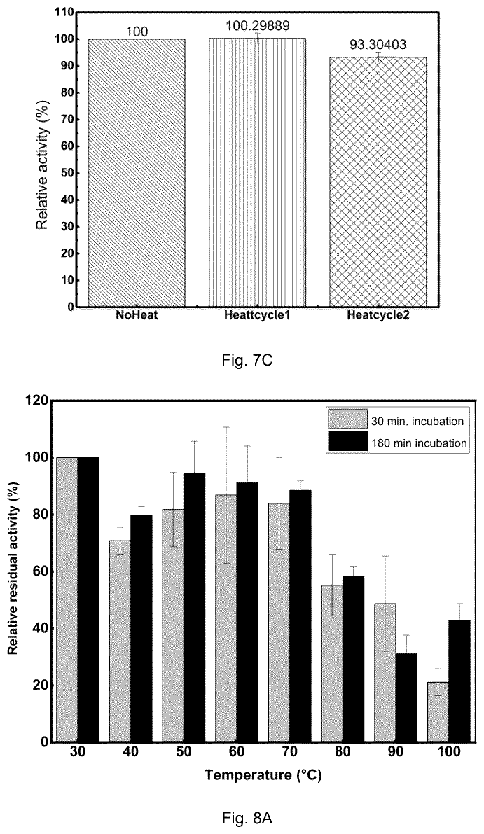

DSC and fluorescence experiment shows that both dAncCA and mAncCA enzymes undergo reversible folding after thermal denaturation. Additionally, temperature-based activity assays revealed that dAncCA and mAncCA remain active after incubation at 100° C. To further analyze the effect of repeated unfolding and refolding on the oligomeric state and activity of these enzymes, repeated heat-cool experiments on both dAncCA, and mAncCA were performed and the activities were compared to the non-heated samples. During a single heat-cool cycle (cycle 1), the enzymes were heated to 100° C. and cooled on ice. In a double heat-cool cycle (cycle 2), the enzymes were heated and cooled twice. Size exclusion chromatography was then used to analyse its effect on the oligomeric state. The SEC profile for dAncCA (FIG. 7A) indicated that the unheated dAncCA exists in equilibrium between monomer and dimer forms, with a higher ratio of dimeric species. The SEC profile of the sample after cycle 1 displayed a decrease in the dimer species, while the monomer form remained stable. After cycle 2, a further decrease in the dimeric form was observed, but no change in the monomeric fraction, confirming that the monomer form is more stable to repeated heating and cooling. Similarly, SEC for mAncCA non-heated, cycle 1 and cycle 2 (data not shown here) reveals that mAncCA elutes through the column existing as a complete monomeric species and the elution profiles were similar for both cycles suggesting that heat-cool cycle did not affect monomer.

Hydratase activity assay was performed on both the enzymes after cycle 1 and cycle 2 and compared the activity to the non-heated sample in order to analyze its effect on the activity of the enzyme. As shown in FIG. 7B, dAncCA showed a decrease in activity after each cycle of heating and cooling, with a 26% and 44% decrease after cycle 1 and cycle 2, respectively. For mAncCA (FIG. 7C), the activity of the sample after cycle 1 was similar to the non-heated sample and only a slight decrease (7%) in the activity was observed after cycle 2. Overall, the results suggested that repeated heating-cooling cycles affect the structural stability and activity of dAncCA but not mAncCA.

Improving the stability of the enzymes while retaining their catalytic efficiency is crucial for their application in industrial processes like post-combustion CO₂ capture. Herein, the ASR method was used to engineer CAs with high thermostability and improved catalytic activity by focusing on the natural enzyme SazCA. AncCA was generated and demonstrated the effectiveness of ASR in producing a highly stable CA ancestor. A comprehensive characterization of the thermostability was performed using DSC, fluorescence and CD spectroscopy, and CO₂ hydration and esterase activity of AncCA. Results showed that AncCA not only exhibits high thermostability of >100° C. and 2.7-fold improved catalytic efficiency but can also refold into a conformation with a functional active site after heat denaturation, which could be beneficial for industrial application. The analyses revealed that the enzyme exists in both dimer and monomer forms (dAncCA and mAncCA), with mAncCA displaying superior thermal stability and activity over repeated heating-cooling cycles.

### Materials and Methods

### Generation of Ancestor Sequences and Phylogenetic Tree Construction

The primary sequence of SazCA was used as a query to identify homologous CA sequences using the protein BLAST (BLASTp) tool available in the National Center for Biotechnology Information (NCBI) resources. Our sequence similarity search resulted in a set of 100 sequences. We used E-values (cut-off 1E-20 used), coverage (>75%), and percentage identity (>30%) to filter and remove redundant sequences from the Blast hits. The selected sequences were aligned using the Multiple Sequence Alignment tool, MAFFT [25] and the alignment was manually curated to remove any gaps. The best evolutionary model that fits the curated data was identified using the ModelFinder program [4] available in the IQTree package. The best model was identified using the Bayesian Information Criterion. A maximum likelihood phylogenetic tree was constructed using the best-fitting model in the IQTree program. All sequences, alignment and trees were analyzed using python, Jalview and FigTree. Node7 node (node7) that was ancestral to SazCA and SspCA and named it AncCA, and the sequence corresponding to this node was predicted using the ASR method implemented in the IQTree package.

### Construction of AncCA and SazCA Gene

The inferred amino acid sequences of ancestor AncCA from IQTree was reverse translated to nucleotide sequences encoding the ancestral proteins. The genes encoding wild-type SazCA; excluding the N-terminal signal peptide sequence was obtained from Uniprot. Both genes were codon optimized for E. coli expression and synthesized by Genscript, USA. The genes were cloned into the plasmid pET-28a between the NheI and XhoI restriction sites, with polyhistidine tag sequence at the N-terminus. The resulting recombinant plasmids were named as pet28α-AncCA and pET28α-SazCA. The constructed plasmids were then transformed into E. coli BL21(DE3) cells by heat shock.

### Protein Expression and Purification

For expression and purification of the resurrected enzyme, AncCA and the extant enzyme, SazCA, the competent E. coli BL21(DE3) cells carrying the respective expression plasmids were inoculated in Luria-Bertani (LB) medium supplemented with 50 mg/mL kanamycin and grown overnight at 37° C. Starter cultures (10 mL) were then used to inoculate 1 Litre LB medium containing 50 mg/mL kanamycin. Cultures were grown until the OD600 reached 0.6 to 0.8 and then induced with 1 mM-isopropyl β-D-1-thiogalactopyranoside (IPTG) and supplemented with 0.5 mM ZnSO4. Cultures were further incubated for 16 h at 20° C. at 200 rpm. The cells were then harvested at 6000×g at 4° C. for 30 min. The cell pellets were resuspended in lysis buffer (50 mM Tris-SO4, 300 mM NaCl pH 8.2) supplemented with protease inhibitor (ThermoScientific) and benzonase endonuclease and disrupted by sonication on ice for 10 min and the soluble fractions were obtained by centrifugation at 23000×g for 30 min. The supernatants were then individually heated at 65° C. for 20 min and centrifuged again at 23000×g for 30 min to remove precipitated proteins. Each enzyme was purified by nickel-immobilized metal affinity chromatography using His resin. The buffer consisted of (50 mM Tris-SO4, 300 mM NaCl, 20 mM imidazole, pH 8.2) as binding buffer and (50 mM Tris-SO4, 300 mM NaCl, 250 mM imidazole, pH 8.2) as elution buffer. Each enzyme was further purified using size exclusion chromatography using Superdex 200 HiLoad 16/60 gel-filtration (GF) column (GE Healthcare) and eluted with (50 mM Tris-SO4, 300 mM NaCl, pH 8.2, 2% glycerol) buffer at 1.0 ml min-1 flow rate. SDS-PAGE was used to assess the purity and homogeneity of each enzyme.

### Differential Scanning Calorimetry

Differential scanning calorimetry (DSC) experiments were performed using Microcal VP-capillary DSC with a cell volume of ˜0.5 ml. dAncCA and SazCA enzyme were buffered in 1×PBS, pH 7.4, at protein concentration of ˜40 mM. The samples were degassed for 30 min before the run. The DSC scans were obtained from 20 to 120° C. The thermograms obtained were buffer-subtracted and baseline-corrected. The calorimetric enthalpies of unfolding for each transition were obtained by integrating the area under the peak for each thermogram. The thermogram were fit to non-two state folding model to obtain the van′t Hoff enthalpies of the transitions. A second scan (scan 2) was performed immediately after cooling the instrument to 20° C. to check the reversibility of the thermal unfolding. The melting temperature (TM) for each transition was obtained from the mid-points of each peak on the DSC thermogram. The results are shown in Table 1.

### Intrinsic Fluorescence Spectroscopy

Intrinsic fluorescence measurements were carried out using a Fluorimeter equipped with a Peltier and a water-jacketed cell holder for temperature control. Fluorescence spectra for 5 μM dAncCA and mAncCA were determined in 20 mM Tris (pH 8.2) and 20 mM NaCl. Both the excitation and emission slit widths were set at 5 nm. Thermal unfolding was observed over a temperature range of 20° C. to 100° C., and the instrument was cooled back to 20° C. to analyse the enzyme refolding. Temperature was controlled externally using a probe of the cell holder. An excitation wavelength of 280 nm was used, and the emission spectra were recorded in the range of 300-400 nm.

### Circular Dichroism (CD)

CD measurements were carried out on JASCO-715 instrument equipped with a peltier thermostated cuvette holder. Enzymes were added at a final concentration of 0.5 mg/mL, 0.2 mg/mL and 0.25 mg/mL for dAncCA, mAncCA and SazCA, respectively. The far-UV CD spectra was collected from 250 to 200 nm using 1 mm path length cuvette. The spectra were recorded over a temperature range of 20° C. to 100° C. with 1° C./min scan rate, and the reversibility of the spectra was analyzed by cooling the instrument back to 20° C. For each sample, three accumulations were averaged to obtain the final spectra. All data collection were performed in 20 mM Tris and 20 mM NaCl buffer, pH 8.2. Spectral units were converted to mean residual ellipticity in degrees and converted to delta epsilon (millidegree M-1 cm-1).

### CO₂ Hydration Activity Assay

The CO₂ hydration activity for AncCAs and SazCA was measured as Wilbur-Anderson units (WAU). The assay was performed based on the decrease in pH due to the catalytic conversion of CO₂ to bicarbonate, monitored using bromothymol blue as an indicator. The indicator turns from blue to yellow upon decrease in the pH from 8.3 to 6.3. The assay was performed on ice, by mixing 1 mL ice cold 25 mM Tris-SO4, pH 8.3, containing bromothymol blue with 2 μL of respective enzyme solution. 1 mL of ice-cold CO₂ saturated water was quickly added to the reaction mixture and the stopwatch was used to record the time required for the reaction mixture to change from blue to yellow. The CO₂ saturated water was prepared by bubbling the CO₂ into water on an ice bath for at least 3 h. The CO₂ hydration activity was calculated as follows: 1WAU=(T0−T)/T, where T0 and T are the time recorded (in seconds) for the colour change transition from blue (pH 8.3) to yellow (pH 6.3) for the uncatalyzed and the catalyzed reaction, respectively.

### Kinetics Parameters Determination Using CO₂ as Substrate

Steady-state Kinetics parameters for CO₂ hydration reaction was determined using a plate-based assay at 0° C. An increasing concentration (5 mM to 65 mM) of CO₂ substrate were prepared from ice-cold CO₂ saturated water (67 mM). Enzyme concentration (83 ng) in a bromothymol blue containing 100 mM Tris (pH 8.3) buffer was used. The reaction was initiated by adding 270 μL of cold CO₂ solution at different concentrations to 30 μL of enzyme-buffer mixture. The initial rates of CO₂ hydration were estimated by monitoring the change in absorbance at 616 nm (color change from blue to yellow). The rates for uncatalyzed reaction were subtracted from the catalyzed reactions to obtain the final rates.

### Temperature Based Hydratase Activity Assay

The effect of temperature on the catalytic activity of the enzymes were assessed by determining the enzyme's residual activity after incubation at various temperatures from 20° C. to 100° C. for different time intervals and comparing it to the activity for non-treated sample at 20° C. Short term (30 min) and long term (180 min) thermostability tests for dAncCA, mAncCA and SazCA were performed using purified enzymes that were diluted to appropriate concentrations. Enzyme solutions (10 μM) were prepared and incubated at different temperatures from 30° C. to 100° C. for the specified times on a heat block. The enzymes were subsequently cooled and centrifuged to remove any precipitation. Enzyme activities for CO₂ were determined using 1 μM final enzyme concentration as described above. Residual activities were calculated relative to the activities for untreated enzymes using equation below.

Residual activity (%)=(activity of heated sample×100)/(activity of non-treated sample)

### Temperature Based Esterase Activity Assay

The β-Nitrophenyl acetate (β-NPA) based esterase activity assay was performed by incubating each enzyme at different temperature ranging from 20° C. to 100° C. for 30 minutes. The reaction mixture containing 20 μL of freshly prepared substrate (15 mM β-NPA in acetone-water mixture) and 270 μL of 100 mM phosphate buffer (pH 7.6) was used and the reaction was initiated by the addition of 10 μL of enzyme solution (10 μM) incubated at various temperatures and the reaction was monitored at 405 nm for 5 min. A Similar uncatalyzed reaction was performed without the enzyme and the rates of the catalyzed reaction were subtracted from the uncatalyzed reaction, to obtain the final activity. All catalyzed and uncatalyzed reactions were performed in triplicate.

### Repeated Heating-Cooling Cycling

To analyze the effect of repeated heating-cooling on the oligomeric state and activity of the enzymes, dAncCA and mAncCA we prepared the samples by heating and cooling these enzymes. No heat samples were non-treated room temperature enzymes to be used as control, Cycle 1 samples were prepared by heating enzymes to 100° C. for 15 minutes and then cooled on ice for an hour and Cycle 2 samples were prepared with enzymes that were heated to 100° C. and cooled on ice for an hour and the process was repeated again to complete two cycles of heating and cooling. All three samples were then applied onto Superdex™ 75 10/300 gel filtration column to analyze the effect of heating and cooling on the oligomeric state of the enzyme. The elution was carried out by monitoring the 280 nm absorbance at a flow rate of 0.3 mL/min. All three samples were further tested for residual hydratase activity using the hydratase assay described above.

### Example 2

Using the ancestral reconstruction methods described in Example 1, the sequence corresponding to nodes 1-6 and 8-11 identified in the phylogenetic tree described in Example 1 (FIG. 1) were also predicted using the ASR method implemented in the IQTree package as described in Example 1. The sequences for Nodes 1-11 are shown in Table 4.

Ancestral enzymes obtained using ASR are predicted to exhibit higher thermostability than their extant forms, and this is often attributed to the prevalent elevated temperatures on Earth during prehistoric times. As such, all nodes identified in the phylogenetic tree described in Example 1 (FIG. 1), including nodes 1-11, are expected to exhibit increased thermostability as compared to SazCA. This expectation is further supported by the data provided herein for nodes 5, 6, and 7, which demonstrate that each of these recombinant carbonic anhydrases exhibit increased/improved thermostability as compared to SazCA.

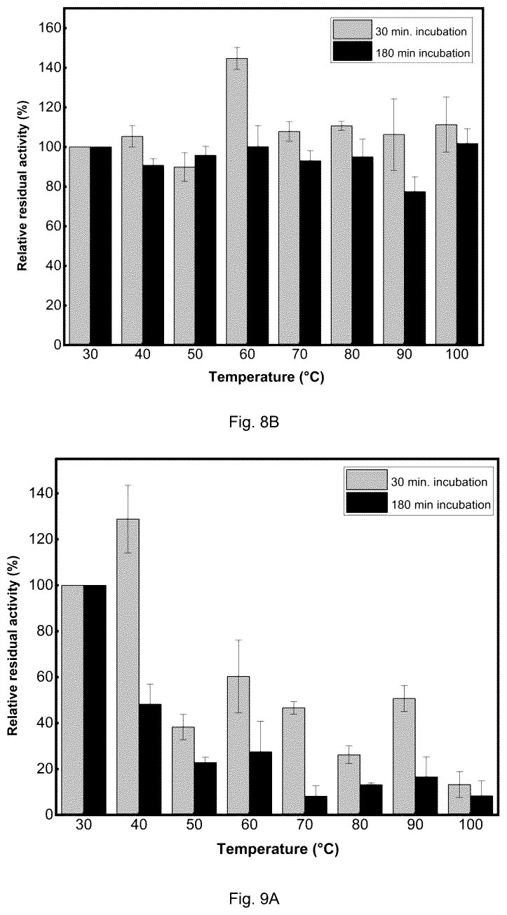

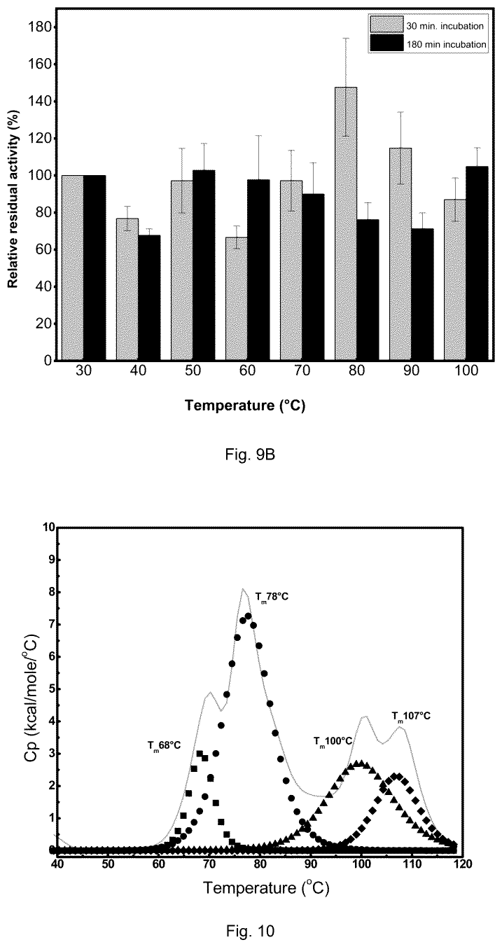

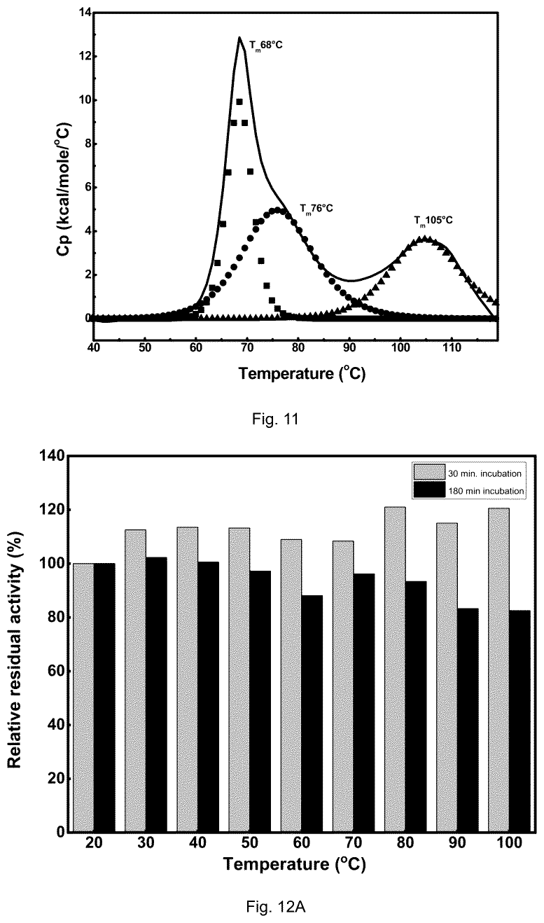

FIGS. 10 and 11 present the differential scanning calorimetry thermograms, showing that node 5 (FIG. 10) and node 6 (FIG. 11) undergo denaturation through distinct transition states, with final T values exceeding 100° C. The relative activity data, FIGS. 8 and 9 (for CO₂ hydration), indicate that the node 5 dimer retains only 20-40% activity, while the node 6 dimer retains 10-20% activity after 30 and 180 minutes of heating at 100° C. In contrast, the monomeric forms of both node 5 and node 6 retain 100% activity under the same conditions, whereas SazCA loses nearly all CO₂ hydration activity (<10%, FIG. 6) at 100° C. These results demonstrate that Node 5 and Node 6 remain stable and active at 100° C. Since the enzyme is likely in equilibrium between dimeric and monomeric states, heat treatment may drive the conversion of dimers into monomers, with monomers displaying activity comparable to that observed at room temperature (20° C.). Collectively, these findings strongly support the conclusion that the two enzymes (Node 5 and Node 6) are highly thermostable.

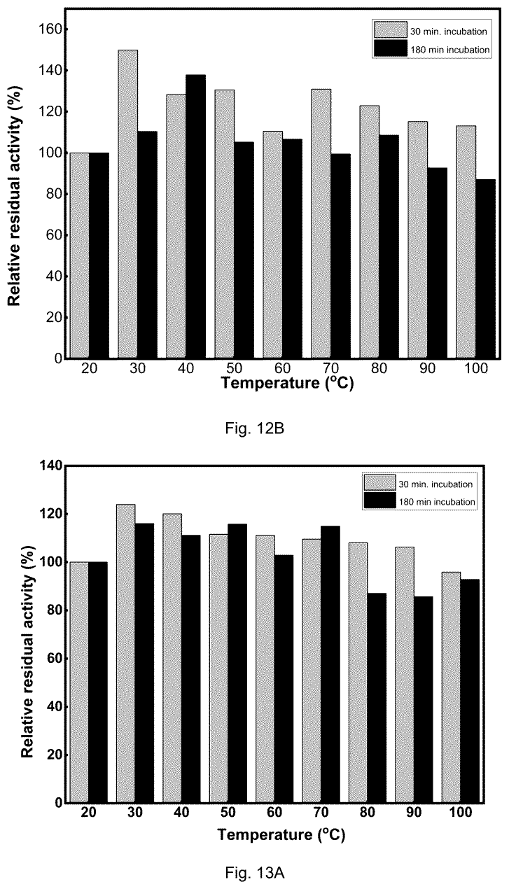

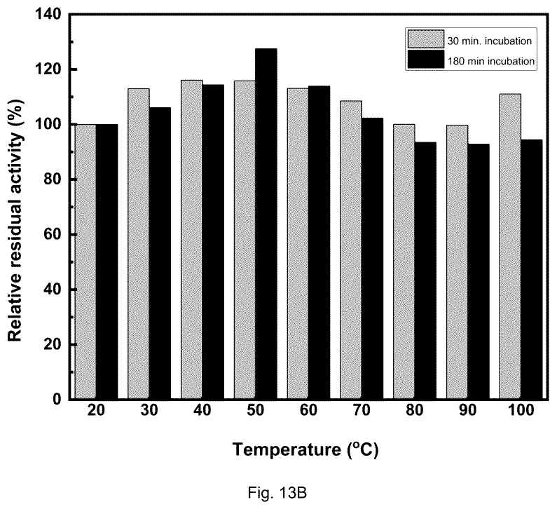

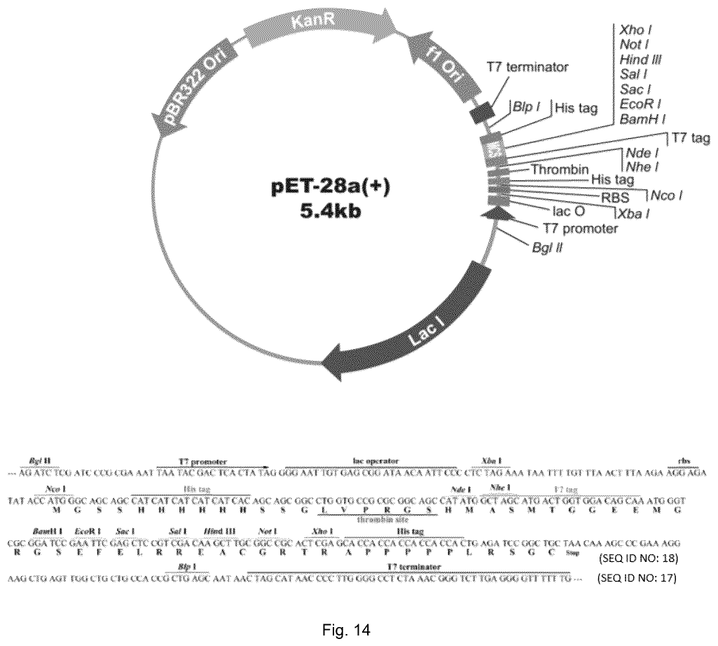

For Esterase activity, FIGS. 12 and 13 demonstrate that both Node 5 and Node 6 maintain almost 100% activity with pNPA substrate, further confirming the high thermostability.

TABLE 4
Amino acid Sequences of Nodes 1-11

| | | SEQ |
|---|---|---|
| Node | | ID |
| # | Amino Acid Sequence | NO |
| 1 | HWGYSGETGPENWAKLTPEFGACAGKNQTPVNLD | 1 |
| | GFIKAELKPLKFNYKAGGSQILNNGHTVQVVYDA | |
| | GSNVVIDGVEYALKQFHFHAPSENQIKGESYPLE | |
| | GHFVHADKDGNLAVVTVMFKEGHANEALESLWTH | |
| | MPAKEGDKILSPANNALKFFPKDHAYYRFSGSLT | |
| | TPPCTEGVRWIVMKKPVFVSKAQIDAFKKVMVHA | |
| | NNRPLQPINAREILG | |
| 2 | HWGYSGETGPENWAKLTPEFGACAGKNQTPVNLS | 2 |
| | GFVKAELKPLKFNYKAGGSQILNNGHTVQVVYDA | |
| | GSNVVIDGVEYALKQFHFHAPSENQIKGESYPLE | |
| | GHFVHADKDGNLAVVTVMFKEGHENEALESLWAH | |
| | MPAKEGDKILSPAFNALKLFPKNHEYYRFSGSLT | |
| | TPPCTEGVRWIVMKKPVFVSKAQIDAFKKVMGHD | |
| | NNRPLQPINAREILE | |
| 3 | HWSYSGETGPEHWAKLTPEFAACAGKNQSPVDIS | 3 |
| | GTVKAELKPLKINYKAAGSEIVNNGHTIQVNYAA | |
| | GSTLVIDGVEFELKQFHFHAPSENTIKGQSYPLE | |
| | AHFVHADKDGNLAVVGVMFKEGKENQALEKLWAH | |
| | MPKEEGEKVLSPAINALALLPKKHEYYRFSGSLT | |
| | TPPCSEGVRWIVMKKPVSVSKEQIDAFKKIMGHD | |
| | NNRPLQPINARTILE | |
| 4 | HWSYSGETGPEHWAKLTPEYFACAGKNQSPVDIS | 4 |
| | GTVKAELKPLKINYKAAGSEIVNNGHTIQVNYAP | |
| | GSTLVIDGTEFELKQFHFHAPSENTVKGQSYPLE | |
| | AHFVHADKDGNLAVIGVMFKEGKENQALEKLWAN | |
| | MPKEEGEKVLSPAINALALLPKKHEYYRFSGSLT | |
| | TPPCSEGVRWIVMKKPVTVSKEQIDAFKKIMGHD | |
| | NNRPVQPINARVILE | |
| 5 | HWSYSGETGPEHWGKLKPEYFMCKGKNQSPVDIN | 5 |
| | GTVEAELKPLKINYKAAGSEIVNNGHTIQVNYEP | |
| | GSYLVVDGIKFELKQFHFHAPSEHTIKGKSYPLE | |
| | AHFVHADKDGNLAVIGVMFKEGKENPELEKLWKN | |
| | MPKEEGKKVLSHAINAYALLPKKKKYYRYSGSLT | |
| | TPPCSEGVRWIVMKKPLTVSKEQIEKFKKIMGHD | |
| | NNRPVQPINARMILE | |
| 6 | HWSYEGETGPEHWGKLKPEYFMCKGKNQSPVDIN | 6 |
| | GTVEAELEPLNINYKAAGSEIVNNGHTIQVNYKE | |
| | DNYLVIDGKKFHLKQFHFHAPSEHTVKGKYYPLE | |
| | MHFVHKDKDGNLAVIGVMFKEGKENPELEKLWKN | |
| | APKEEGKKVLDSSINMNALLPKKKDYYRYSGSLT | |
| | TPPCSEGVRWIVLKKPITVSKEQIEKFKKIMGHD | |
| | NNRPVQPINARMILE | |
| 7 | HWSYEGENGPEHWAKLKPEYFWCKLKNQSPVDIS | 7 |
| | DKVKAKLEKLNINYKKANSEIVNNGHTIQVNVKE | |
| | DNTLNIKGKKYHLKQFHFHAPSEHTVEGKYYPLE | |
| | MHFVHKDKDGNIAVIGVMFKEGKANPELDKVFKN | |
| | APKEEGEKVLDGSINLNALLPKDKNYYTYSGSLT | |
| | TPPCTEGVRWIVLKQPITVSKQQIEKFKSIMKHD | |
| | NNRPVQPINSRYILE | |
| 8 | HWSYEGENGPENWAKLNPEYFWCNLKNQSPVDIS | 8 |
| | DKVHAKLEKLNINYNKANPEIVNNGHTIQVNVLE | |
| | DFKLNIKGKEYHLKQFHFHAPSEHTVNGKYYPLE | |
| | MHLVHKDKDGNIAVIGVFFKEGKANPELDKVFKN | |
| | ALKEEGSKVFDGSINLNALLPPVKNYYTYSGSLT | |
| | TPPCTEGVLWIVLKQPITASKQQIELFKSIMKHN | |
| | NNRPTQPINSRYILE | |
| 9 | HWSYHGETGPEHWGDLKDEYIMCKGKNQSPVDIN | 9 |
| | RIVEAELKKLKINYKSGGSSIVNNGHTIKVSYEP | |
| | GSYIVVDGIKFELKQFHFHAPSEHKIKGKSYPFE | |
| | AHFVHADKDGNLAVIGVVFKEGKENPVIEKLWKN | |
| | LPSEVGKKVLAHKINAYDLLPKKKKYYRYSGSLT | |
| | TPPCSEGVRWIVMKEELEMSKEQIEKFRKLMGGD | |
| | TNRPVQPLNARMIME | |
| 10 | HWSYHGETGPQHWGDLKNEYIMCKGKNQSPVDIS | 10 |
| | RIVEAELKKLKINYSSGGSSITNNGHTIKVSYEP | |
| | GSYIVVDGIRFELKQFHFHAPSEHKIKGKSYPFE | |
| | AHFVHADKDGNLAVIGVIFKEGKKNPVIEKIWKN | |
| | LPSEAGKTVLAHKINAYDLLPKKKKYYRYSGSLT | |
| | TPPCSEGVRWIVMKEEMELSKEQIEKFRKLMGGD | |
| | TNRPVQPLNARMIME | |
| 11 | NWSYHGETGPEHWGDLKDEYIMCKGKNQSPVDIN | 11 |
| | RIVEAELKDLKINYKAGATSIVNNGHTIKVSYEP | |
| | GSYIVVDGIKFELKQFHFHAPSEHKIKGKSYPFE | |
| | AHFVHADKDGNLAVIGVVFKEGKENPVIEKLWKN | |
| | LPSEVGKKVLAHKINAYDLLPKKKKYYRYSGSLT | |
| | TPPCSEGVRWIVMKEELEMSKEQIEKFRKLMGGD | |
| | TNRPVQPLNARMIME | |

TABLE 5
Sequence Alignments of SazCA and Nodes 1-11
SEQ ID NO

| | | |
|---|---|---|
| 7 | ----------------------------------HWSYEGENGPEHWAKLKPEYFWCK-L | 25 |
| 13 | MKK----FILSILSLSIVSIAGEHAILQKNAEVHHWSYEGENGPENWAKLNPEYFWCN-L | 55 |
| 8 | ----------------------------------HWSYEGENGPENWAKLNPEYFWCN-L | 25 |
| 2 | ----------------------------------HWGYSGETGPENWAKLTPEFGACA-G | 25 |
| 1 | ----------------------------------HWGYSGETGPENWAKLTPEFGACA-G | 25 |
| 10 | ----------------------------------HWSYHGETGPQHWGDLKNEYIMCK-G | 25 |
| 11 | ----------------------------------NWSYHGETGPEHWGDLKDEYIMCK-G | 25 |
| 9 | ----------------------------------HWSYHGETGPEHWGDLKDEYIMCK-G | 25 |
| 4 | ----------------------------------HWSYSGETGPEHWAKLTPEYFACA-G | 25 |
| 3 | ----------------------------------HWSYSGETGPEHWAKLTPEFAACA-G | 25 |
| 6 | ----------------------------------HWSYEGETGPEHWGKLKPEYFMCK-G | 25 |
| 5 | ----------------------------------HWSYSGETGPEHWGKLKPEYFMCK-G | 25 |
| 7 | KNQSPVDISDK--VKAKLEKLNINYKKA-NSEIVNNGHTIQVNVKEDNTLNIKGKKYHLK | 82 |
| 13 | KNQSPVDISDNYKVHAKLEKLHINYNKAVNPEIVNNGHTIQVNVLEDFKLNIKGKEYHLK | 115 |
| 8 | KNQSPVDISDK--VHAKLEKLNINYNKA-NPEIVNNGHTIQVNVLEDFKLNIKGKEYHLK | 82 |
| 2 | KNQTPVNLSGF--VKAELKPLKFNYKAG-GSQILNNGHTVQVVYDAGSNVVIDGVEYALK | 82 |
| 1 | KNQTPVNLDGF--IKAELKPLKFNYKAG-GSQILNNGHTVQVVYDAGSNVVIDGVEYALK | 82 |
| 10 | KNQSPVDISRI--VEAELKKLKINYSSG-GSSITNNGHTIKVSYEPGSYIVVDGIRFELK | 82 |
| 11 | KNQSPVDINRI--VEAELKDLKINYKAG-ATSIVNNGHTIKVSYEPGSYIVVDGIKFELK | 82 |
| 9 | KNQSPVDINRI--VEAELKKLKINYKSG-GSSIVNNGHTIKVSYEPGSYIVVDGIKFELK | 82 |
| 4 | KNQSPVDISGT--VKAELKPLKINYKAA-GSEIVNNGHTIQVNYAPGSTLVIDGTEFELK | 82 |
| 3 | KNQSPVDISGT--VKAELKPLKINYKAA-GSEIVNNGHTIQVNYAAGSTLVIDGVEFELK | 82 |
| 6 | KNQSPVDINGT--VEAELEPLNINYKAA-GSEIVNNGHTIQVNYKEDNYLVIDGKKFHLK | 82 |
| 5 | KNQSPVDINGT--VEAELKPLKINYKAA-GSEIVNNGHTIQVNYEPGSYLVVDGIKFELK | 82 |
| 7 | QFHFHAPSEHTVEGKYYPLEMHFVHKDKDGNIAVIGVMFKEGKANPELDKVFKNAPKEEG | 142 |
| 13 | QFHFHAPSEHTVNGKYYPLEMHLVHKDKDGNIAVIGVFFKEGKANPELDKVFKNALKEEG | 175 |
| 8 | QFHFHAPSEHTVNGKYYPLEMHLVHKDKDGNIAVIGVFFKEGKANPELDKVFKNALKEEG | 142 |
| 2 | QFHFHAPSENQIKGESYPLEGHFVHADKDGNLAVVTVMFKEGHENEALESLWAHMPAKEG | 142 |
| 1 | QFHFHAPSENQIKGESYPLEGHFVHADKDGNLAVVTVMFKEGHANEALESLWTHMPAKEG | 142 |
| 10 | QFHFHAPSEHKIKGKSYPFEAHFVHADKDGNLAVIGVIFKEGKKNPVIEKIWKNLPSEAG | 142 |
| 11 | QFHFHAPSEHKIKGKSYPFEAHFVHADKDGNLAVIGVVFKEGKENPVIEKLWKNLPSEVG | 142 |
| 9 | QFHFHAPSEHKIKGKSYPFEAHFVHADKDGNLAVIGVVFKEGKENPVIEKLWKNLPSEVG | 142 |
| 4 | QFHFHAPSENTVKGQSYPLEAHFVHADKDGNLAVIGVMFKEGKENQALEKLWANMPKEEG | 142 |
| 3 | QFHFHAPSENTIKGQSYPLEAHFVHADKDGNLAVVGVMFKEGKENQALEKLWAHMPKEEG | 142 |
| 6 | QFHFHAPSEHTVKGKYYPLEMHFVHKDKDGNLAVIGVMFKEGKENPELEKLWKNAPKEEG | 142 |
| 5 | QFHFHAPSEHTIKGKSYPLEAHFVHADKDGNLAVIGVMFKEGKENPELEKLWKNMPKEEG | 142 |
| 7 | EKV-LDGSINLNALLPKDKNYYTYSGSLTTPPCTEGVRWIVLKQPITVSKQQIEKFKSIM | 201 |
| 13 | SKV-FDGSININALLPPVKNYYTYSGSLTTPPCTEGVLWIVLKQPITASKQQIELFKSIM | 234 |
| 8 | SKV-FDGSINLNALLPPVKNYYTYSGSLTTPPCTEGVLWIVLKQPITASKQQIELFKSIM | 201 |
| 2 | DKI-LSPAFNALKLFPKNHEYYRFSGSLTTPPCTEGVRWIVMKKPVFVSKAQIDAFKKVM | 201 |
| 1 | DKI-LSPANNALKFFPKDHAYYRFSGSLTTPPCTEGVRWIVMKKPVFVSKAQIDAFKKVM | 201 |
| 10 | KTV-LAHKINAYDLLPKKKKYYRYSGSLTTPPCSEGVRWIVMKEEMELSKEQIEKFRKLM | 201 |
| 11 | KKV-LAHKINAYDLLPKKKKYYRYSGSLTTPPCSEGVRWIVMKEELEMSKEQIEKFRKLM | 201 |
| 9 | KKV-LAHKINAYDLLPKKKKYYRYSGSLTTPPCSEGVRWIVMKEELEMSKEQIEKFRKLM | 201 |
| 4 | EKV-LSPAINALALLPKKHEYYRFSGSLTTPPCSEGVRWIVMKKPVTVSKEQIDAFKKIM | 201 |
| 3 | EKV-LSPAINALALLPKKHEYYRFSGSLTTPPCSEGVRWIVMKKPVSVSKEQIDAFKKIM | 201 |
| 6 | KKV-LDSSINMNALLPKKKDYYRYSGSLTTPPCSEGVRWIVLKKPITVSKEQIEKFKKIM | 201 |
| 5 | KKV-LSHAINAYALLPKKKKYYRYSGSLTTPPCSEGVRWIVMKKPLTVSKEQIEKFKKIM | 201 |
| 7 | KHDNNRPVQPINSRYILE-- | 219 |
| 13 | KHNNNRPTQPINSRYILESN | 254 |
| 8 | KHNNNRPTQPINSRYILE-- | 219 |
| 2 | GHDNNRPLQPINAREILE-- | 219 |
| 1 | VHANNRPLQPINAREILG-- | 219 |
| 10 | GGDTNRPVQPLNARMIME-- | 219 |
| 11 | GGDTNRPVQPLNARMIME-- | 219 |
| 9 | GGDTNRPVQPLNARMIME-- | 219 |
| 4 | GHDNNRPVQPINARVILE-- | 219 |
| 3 | GHDNNRPLQPINARTILE-- | 219 |
| 6 | GHDNNRPVQPINARMILE-- | 219 |
| 5 | GHDNNRPVQPINARMILE-- | 219 |

TABLE 6
Sequence Identifies of Nodes 1-11 as compared to each other

| | |
|---|---|
| Node8 vs Node7 | 196/219 (89.5%) |
| Node8 vs Node6 | 165/219 (75.3%) |
| Node8 vs Node10 | 123/219 (56.2%) |
| Node8 vs Node11 | 123/219 (56.2%) |
| Node8 vs Node9 | 125/219 (57.1%) |
| Node8 vs Node5 | 147/219 (67.1%) |
| Node8 vs Node4 | 144/219 (65.8%) |
| Node8 vs Node3 | 138/219 (63.0%) |
| Node8 vs Node2 | 119/219 (54.3%) |
| Node8 vs Node1 | 116/219 (53.0%) |
| Node7 vs Node6 | 183/219 (83.6%) |
| Node7 vs Node10 | 135/219 (61.6%) |
| Node7 vs Node11 | 136/219 (62.1%) |
| Node7 vs Node9 | 139/219 (63.5%) |
| Node7 vs Node5 | 163/219 (74.4%) |
| Node7 vs Node4 | 158/219 (72.1%) |
| Node7 vs Node3 | 151/219 (68.9%) |
| Node7 vs Node2 | 128/219 (58.4%) |
| Node7 vs Node1 | 125/219 (57.1%) |
| Node6 vs Node10 | 158/219 (72.1%) |
| Node6 vs Node11 | 164/219 (74.9%) |
| Node6 vs Node9 | 166/219 (75.8%) |
| Node6 vs Node5 | 196/219 (89.5%) |
| Node6 vs Node4 | 179/219 (81.7%) |
| Node6 vs Node3 | 172/219 (78.5%) |
| Node6 vs Node2 | 144/219 (65.8%) |
| Node6 vs Node1 | 138/219 (63.0%) |
| Node10 vs Node11 | 201/219 (91.8%) |
| Node10 vs Node9 | 206/219 (94.1%) |
| Node10 vs Node5 | 176/219 (80.4%) |
| Node10 vs Node4 | 154/219 (70.3%) |
| Node10 vs Node3 | 150/219 (68.5%) |
| Node10 vs Node2 | 131/219 (59.8%) |
| Node10 vs Node1 | 125/219 (57.1%) |
| Node11 vs Node9 | 214/219 (97.7%) |
| Node11 vs Node5 | 183/219 (83.6%) |
| Node11 vs Node4 | 157/219 (71.7%) |
| Node11 vs Node3 | 153/219 (69.9%) |
| Node11 vs Node2 | 133/219 (60.7%) |
| Node11 vs Node1 | 127/219 (58.0%) |
| Node9 vs Node5 | 185/219 (84.5%) |
| Node9 vs Node4 | 159/219 (72.6%) |
| Node9 vs Node3 | 155/219 (70.8%) |
| Node9 vs Node2 | 135/219 (61.6%) |
| Node9 vs Node1 | 129/219 (58.9%) |
| Node5 vs Node4 | 192/219 (87.7%) |
| Node5 vs Node3 | 186/219 (84.9%) |
| Node5 vs Node2 | 156/219 (71.2%) |
| Node5 vs Node1 | 150/219 (68.5%) |
| Node4 vs Node3 | 209/219 (95.4%) |
| Node4 vs Node2 | 174/219 (79.5%) |
| Node4 vs Node1 | 165/219 (75.3%) |
| Node3 vs Node2 | 181/219 (82.6%) |
| Node3 vs Node1 | 172/219 (78.5%) |
| Node2 vs Node1 | 208/219 (95.0%) |

TABLE 7
Sequence Identity of SazCA (SEQ ID NO: 13) with Nodes 1-11.

| | |
|---|---|
| SazCA vs Node8 | 217/254 (85.4%) |
| SazCA vs Node7 | 194/254 (76.4%) |
| SazCA vs Node6 | 166/254 (65.4%) |
| SazCA vs Node10 | 123/254 (48.4%) |
| SazCA vs Node11 | 126/254 (49.6%) |
| SazCA vs Node9 | 127/254 (50.0%) |
| SazCA vs Node5 | 149/254 (58.7%) |
| SazCA vs Node4 | 145/254 (57.1%) |
| SazCA vs Node3 | 139/254 (54.7%) |
| SazCA vs Node2 | 121/254 (47.6%) |
| SazCA vs Node1 | 119/254 (46.9%) |

TABLE 8
Nucleotide Sequences of Nodes 5-7

| | | SEQ |
|---|---|---|
| Node | | ID |
| # | Nucleotide Sequence | NO |
| Node | CACTGGTCATATAGTGGAGAAACAGGGCCCGAGC | 14 |
| 5 | ATTGGGGTAAGCTTAAGCCGGAATACTTCATGTG | |
| | CAAAGGTAAGAACCAGAGCCCGGTTGACATCAAC | |
| | GGCACCGTGGAAGCAGAACTCAAGCCACTGAAGA | |
| | TCAATTATAAGGCGGCAGGTTCGGAGATCGTCAA | |
| | TAACGGCCACACCATTCAAGTAAACTACGAACCG | |
| | GGTTCCTACCTGGTCGTGGACGGCATTAAATTCG | |
| | AGCTGAAGCAGTTTCATTTCCACGCGCCAAGCGA | |
| | GCACACCATCAAGGGCAAGTCTTACCCGTTGGAA | |
| | GCGCATTTTGTTCACGCGGATAAAGACGGAAACC | |
| | TGGCTGTGATCGGCGTGATGTTCAAAGAGGGCAA | |
| | AGAGAATCCGGAACTGGAGAAACTGTGGAAAAAC | |
| | ATGCCGAAGGAGGAAGGTAAGAAAGTTCTGAGCC | |
| | ATGCGATCAACGCTTATGCTCTGTTACCGAAAAA | |
| | AAAGAAATATTACCGCTATAGCGGTTCTCTGACG | |
| | ACTCCGCCTTGTAGCGAGGGTGTGCGTTGGATTG | |
| | TTATGAAAAAGCCGTTGACCGTTTCCAAAGAACA | |
| | AATTGAAAAATTTAAGAAGATCATGGGTCACGAT | |
| | AATAATCGTCCGGTTCAGCCGATTAACGCCCGTA | |
| | TGATTTTGGAGTAA | |
| Node | CACTGGTCATATGAAGGAGAGACAGGGCCCGAGC | 15 |
| 6 | ATTGGGGTAAGCTCAAGCCGGAATATTTCATGTG | |
| | CAAAGGTAAGAACCAGTCGCCGGTTGATATCAAT | |
| | GGCACCGTGGAAGCGGAGTTGGAGCCGTTGAATA | |
| | TTAACTACAAAGCGGCGGGTTCTGAGATCGTTAA | |
| | TAACGGCCATACCATTCAAGTGAACTATAAAGAG | |
| | GACAACTACCTGGTTATCGACGGCAAGAAGTTCC | |
| | ACCTGAAGCAATTTCACTTCCACGCTCCGAGCGA | |
| | GCACACTGTAAAAGGCAAGTATTACCCGCTGGAA | |
| | ATGCACTTTGTTCATAAAGACAAAGATGGTAATC | |
| | TGGCAGTTATTGGTGTGATGTTTAAAGAAGGCAA | |
| | GGAAAATCCGGAATTAGAGAAGCTTTGGAAAAAC | |
| | GCACCGAAAGAGGAAGGTAAAAAGGTTCTGGATA | |
| | GCAGCATCAACATGAATGCGTTGCTGCCGAAAAA | |
| | GAAGGACTACTACCGCTATAGCGGTTCCCTGACC | |
| | ACGCCACCGTGTTCCGAGGGCGTGCGTTGGATTG | |
| | TGCTGAAGAAACCGATTACCGTCAGCAAAGAGCA | |
| | GATTGAAAAATTCAAAAAGATCATGGGTCATGAT | |
| | AACAACCGTCCGGTCCAGCCTATCAACGCCCGTA | |
| | TGATCCTGGAATAA | |
| Node | CACTGGTCATATGAGGGAGAAAATGGGCCCGAGC | 16 |
| 7 | ATTGGGCAAAATTGAAGCCGGAGTACTTCTGGTG | |
| | TAAATTGAAGAACCAGAGCCCGGTCGACATCAGC | |
| | GATAAGGTGAAAGCCAAATTGGAGAAGTTAAATA | |
| | TCAACTATAAAAAGGCGAATTCCGAGATCGTGAA | |
| | TAACGGCCATACGATTCAGGTTAATGTTAAAGAA | |
| | GATAATACCCTGAACATCAAAGGTAAGAAATACC | |
| | ATCTGAAGCAATTTCACTTCCACGCTCCGAGCGA | |
| | GCACACGGTTGAAGGCAAATATTACCCGCTGGAA | |
| | ATGCATTTCGTGCACAAAGACAAGGACGGTAATA | |
| | TTGCTGTTATTGGTGTTATGTTTAAGGAGGGCAA | |
| | AGCGAACCCGGAACTGGATAAGGTCTTTAAAAAC | |
| | GCGCCGAAAGAGGAGGGTGAAAAAGTGCTGGACG | |
| | GCTCCATCAACCTCAATGCGCTGCTGCCGAAAGA | |
| | CAAGAACTACTACACCTATAGCGGTTCTCTGACC | |
| | ACTCCGCCTTGCACCGAAGGCGTGCGTTGGATTG | |
| | TTCTTAAGCAACCGATCACCGTGAGCAAACAACA | |
| | GATTGAAAAGTTCAAGTCGATCATGAAGCACGAT | |
| | AACAACCGCCCAGTTCAGCCGATTAACTCTCGTT | |
| | ATATCCTGGAATAA | |

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples but should be given the broadest interpretation consistent with the description as a whole.

### REFERENCES

[1]G. T. Rochelle, “Amine Scrubbing for CO₂ Capture,” Science, vol. 325, no. 5948, pp. 1652-1654, 2009, doi: 10.1126/science.1176731.
[2]A. Di Fiore, V. Alterio, S. M. Monti, G. De Simone, and K. D'Ambrosio, “Thermostable carbonic anhydrases in biotechnological applications,” International Journal of Molecular Sciences, vol. 16, no. 7, pp. 15456-15480, 2015.
[3]E. A. Gaucher, J. M. Thomson, M. F. Burgan, and S. A. Benner, “Inferring the palaeoenvironment of ancient bacteria on the basis of resurrected proteins,” Nature, vol. 425, no. 6955, pp. 285-288, September 2003, doi: 10.1038/nature01977.
[4]“Kalyaanamoorthy, S., Minh, B. Q., Wong, T. K., Von Haeseler, A., & Jermiin, L. S. (2017). ModelFinder: fast model selection for accurate phylogenetic estimates. Nature methods, 14(6), 587-589.”.
[5]“De Luca, V., Vullo, D., Scozzafava, A., Carginale, V., Rossi, M., Supuran, C. T., & Capasso, C. (2013). An α-carbonic anhydrase from the thermophilic bacterium Sulphurihydrogenibium azorense is the fastest enzyme known for the CO₂ hydration reaction. Bioorganic & medicinal chemistry, 21(6), 1465-1469.”.
[6]S. Ghaedizadeh, M. Zeinali, B. Dabirmanesh, B. Rasekh, K. Khajeh, and A. M. Banaei-Moghaddam, “Rational design engineering of a more thermostable Sulfurihydrogenibium yellowstonense carbonic anhydrase for potential application in carbon dioxide capture technologies,” Biochimica et Biophysica Acta (BBA)-Proteins and Proteomics, vol. 1872, no. 1, p. 140962, 2024.

1. A recombinant carbonic anhydrase polypeptide comprising:
an amino acid sequence having one or more amino acid modification as compared to SEQ ID NO: 13, wherein amino acid residues W32, Y34, G36, E37, G39, P40, W43, L46, E49, C53, K56, N57, Q58, P60, V61, A71, L73, L76, N79, Y80, 188, N90, N91, G92, H93, T94, V97, G109, L114, K115, Q116, F117, H118, F119, H120, A121, P122, S123, E124, G129, Y132, P133, E135, H137, V139, H140, D142, K143, D144, G145, N146, A148, V149, V152, F154, K155, E156, G157, N160, G175, N184, P190, Y195, Y196, S199, G200, D201, L202, T203, T24, P205, P206, C207, E209, G210, V211, W213, I214, V215, K217, S223, K224, Q226, I227, F230, M234, N239, R240, P241, Q243, P244, N246, R248, and/or I250, are unmodified; and/or
an amino acid sequence of SEQ ID NO: 1 or a sequence having at least about 55% sequence identity to SEQ ID NO: 1,
an amino acid sequence of SEQ ID NO: 2 or a sequence having at least about 57% sequence identity to SEQ ID NO: 2,
an amino acid sequence of SEQ ID NO: 3 or a sequence having at least about 65% sequence identity to SEQ ID NO: 3,
an amino acid sequence of SEQ ID NO: 4 or a sequence having at least about 67% sequence identity to SEQ ID NO: 4,
an amino acid sequence of SEQ ID NO: 5 or a sequence having at least about 76% sequence identity to SEQ ID NO: 5,
an amino acid sequence of SEQ ID NO: 6 or a sequence having at least about 69% sequence identity to SEQ ID NO: 6,
an amino acid sequence of SEQ ID NO: 7 or a sequence having at least about 73% sequence identity to SEQ ID NO: 7,
an amino acid sequence of SEQ ID NO: 8 or a sequence having at least about 86% sequence identity to SEQ ID NO: 8,
an amino acid sequence of SEQ ID NO: 9 or a sequence having at least about 87% sequence identity to SEQ ID NO: 9,
an amino acid sequence of SEQ ID NO: 10 or a sequence having at least about 93% sequence identity to SEQ ID NO: 10, or
an amino acid sequence of SEQ ID NO: 11 or a sequence having at least about 85% sequence identity to SEQ ID NO: 111.

2. The recombinant carbonic anhydrase polypeptide of claim 1, comprising an amino acid sequence of:
(SEQ ID NO: 12)
X₁WX₂YX₃GEX₄GPX₅X₆WX₇X₈LX₉X₁₀EX₁₁X₁₂X₁₃CX₁₄X₁₅
KNQX₁₆PVX₁₇X₁₈X₁₉X₂₀X₂₁X₂₂X₂₃AX₂₄LX₂₅X₂₆LX₂₇
X₂₈NYX₂₉X₃₀X₃₁X₃₂X₃₃X₃₄IX₃₅NNGHTX₃₆X₃₇VX₃₈X₃₉
X₄₀X₄₁X₄₂X₄₃X₄₄X₄₅X₄₆X₄₇X₄₈GX₄₉X₅₀X₅₁X₅₂
LKQFHFHAPSEX₅₃X₅₄X₅₅X₅₆GX₅₇X₅₈YPX₅₉EX₆₀HX₆₁
VHX₆₂DKDGNX₆₃AVX₆₄X₆₅VX₆₆FKEGX₆₇X₆₈NX₆₉X₇₀X₇₁
X₇₂X₇₃X₇₄X₇₅X₇₆X₇₇X₇₈X₇₉X₈₀X₈₁X₈₂GX₈₃X₈₄X₈₅
X₈₆X₈₇X₈₈X₈₉X₉₀NX₉₁X₉₂X₉₃X₉₄X₉₅PX₉₆X₉₇X₉₈X₉₉
YYX₁₀₀X₁₀₁SGSLTTPPCX₁₀₂EGVX₁₀₃WIVX₁₀₄KX₁₀₅
X₁₀₆X₁₀₇X₁₀₈X₁₀₉SKX₁₁₀QIX₁₁₁X₁₁₂FX₁₁₃X₁₁₄X₁₁₅
MX₁₁₆X₁₁₇X₁₁₈X₁₁₉NRPX₁₂₀QPX₁₂₁NX₁₂₂RX₁₂₃IX₁₂₄
X₁₂₅,
wherein: X₁ is H or N; X₂ is G or S; X₃ is S, H or E; X₄ is T or N; X₅ is E or Q; X₆ is N or H; X₇ is A or G; X₈ is K or D; X₉ is T, N or K; X₁₀ is P, N or D; X₁ is F or Y; X₁₂ is G, F, I or A; X₁₃ is A, W or M; X₁₄ is A, N or K; X₁₅ is G or L; X₁₆ is T or S; X₁₇ is N or D; X₁₈ is L or I; X₁₉ is S, D or N; X₂₀ is G, D or R; X₂₁ is F, K, I or T; X₂₂ is V or I; X₂₃ is K, H or E; X₂₄ is E or K; X₂₅ is K or E; X₂₆ is P, K, or D; X₂₇ is K or N; X₂₈ is F or I; X₂₉ is K, N or S; X₃₀ is A, K or S; X₃₁ is G or A; X₃₂ is G, N or A; X₃₃ is S, T or P; X₃₄ is Q, E or S; X₃₅ is L, V, or T; X₃₆ is V or I; X₃₇ is Q or K; X₃₈ is V, N or S; X₃₉ is Yor V; X₄₀ is D, L, K, E or A; X₄₁ is A, E or P; X₄₂ is G or D; X₄₃ is S, F, or N; X₄₄ is N, K, T or Y; X₄₅ is V, L or I; X₄₆ is V or N; X₄₇ is I or V; X₄₈ is D or K; X₄₉ is V, K, I or T; X₅₀ is E, K or R; X₅₁ is Yor F; X₅₂ is A, H or E; X₅₃ is H or N; X₅₄ is Q, T or K; X₅₅ is I or V; X₅₆ is K, N or E; X₅₇ is E, K, or Q; X₅₈ is S or Y; X₅₉ is L or F; X₆₀ is G, M or A; X₆₁ is F or L; X₆₂ is A or K; X₆₃ is L or I; X₆₄ is V or I; X₆₅ is T or G; X₆₆ is M, F, I or V; X₆₇ is H or K; X₆₈ is E, K or A; X₆₉ is E, P, or Q; X₇₀ is A, E or V; X₇₁ is L or I; X₇₂ is E or D; X₇₃ is S or K; X₇₄ is L, V or I; X₇₅ is W or F; X₇₆ is A, T or K; X₇₇ is H or N; X₇₈ is M, A or L; X₇₉ is P or L; X₈₀ is A, K or S; X₈₁ is K or E; X₈₂ is E, A or V; X₈₃ is D, S, E or K; X₈₄ is K or T; X₈₅ is I or V; X₈₆ is L or F; X₈₇ is S, D, or A; X₈₈ is P, G, H or S; X₈₉ is A, S or K; X₉₀ is F, N or I; X₉₁ is A, L, or M; X₉₂ is L, N or Y; X₉₃ is K, A or D; X₉₄ is L or F; X₉₅ is F or L; X₉₆ is K or P; X₉₇ is N, D, V or K; X₉₈ is H or K; X₉₉ is E, A, N, K or D; X₁₀₀ is R or T; X₁₀₁ is F or Y; X₁₀₂ is T or S; X₁₀₃ is R or L; X₁₀₄ is M or L; X₁₀₅ is K, Q, or E; X₁₀₆ is P or E; X₁₀₇ is V, I, M or L; X₁₀₈ is F, T, E or S; X₁₀₉ is V, A, L or M; X₁₁₀ is A, Q or E; X₁₁₁ is D or E; X₁₁₂ is A, L or K; X₁₃ is K or R; X₁₁₄ is K or S; X₁₁₅ is V, I or L; X₁₁₆ is G, V or K; X₁₁₇ is H or G; X₁₁₈ is D, A, or N; X₁₁₉ is N or T; X₁₂₀ is L, T, or V; X₁₂₁ is I or L; X₁₂₂ is A or S; X₁₂₃ is E, Y, M, V, or T; X₁₂₄ is L or M; and/or X₁₂₅ is E or G.

3. The recombinant carbonic anhydrase polypeptide of claim 1, wherein the amino acid sequence comprises any one of SEQ ID NOs: 1-11.

4. The recombinant carbonic anhydrase polypeptide of claim 1, wherein the amino acid sequence comprises any one of SEQ ID NOs: 5-7.

5. The recombinant carbonic anhydrase polypeptide of claim 1, wherein the amino acid comprises SEQ ID NO: 5.

6. The recombinant carbonic anhydrase polypeptide of claim 1, wherein the amino acid comprises SEQ ID NO: 6.

7. The recombinant carbonic anhydrase polypeptide of claim 1, wherein the amino acid comprises SEQ ID NO: 7.

8. The recombinant carbonic anhydrase polypeptide of any one of claim 1, wherein the recombinant carbonic anhydrase polypeptide comprises increased hydratase activity as compared to wild type SazCA, optionally wherein the recombinant carbonic anhydrase polypeptide comprises at least about 2-fold higher hydratase activity as compared to wild type SazCA; and/or wherein the recombinant carbonic anhydrase polypeptide renatures after denaturation.

9. The recombinant carbonic anhydrase polypeptide of any one of claim 1, wherein the recombinant carbonic anhydrase polypeptide comprises increased thermostability as compared to wild type SazCA, optionally wherein the recombinant carbonic anhydrase polypeptide comprises at least about 10° C.-15° C. increase in its temperature for unfolding as compared to wild type SazCA.

10. The recombinant carbonic anhydrase polypeptide of any one of claim 1, wherein the recombinant carbonic anhydrase polypeptide is a monomer.

11. The recombinant carbonic anhydrase polypeptide of claim 1, wherein the recombinant carbonic anhydrase polypeptide is a dimer.

12. The recombinant carbonic anhydrase polypeptide of claim 1, is a purified polypeptide.

13. An isolated nucleic acid encoding the recombinant carbonic anhydrase polypeptide of any one of claim 1.

14. A vector or expression cassette encoding the recombinant carbonic anhydrase polypeptide of claim 1 or an isolated nucleic acid encoding the recombinant carbonic anhydrase polypeptide.

15. A cell comprising the vector or expression cassette of claim 14.

16. A composition comprising the recombinant carbonic anhydrase polypeptide of claim 1, an isolated nucleic acid encoding the recombinant carbonic anhydrase polypeptide, the vector or expression cassette encoding the isolated nucleic acid, and/or the cell comprising the vector, and optionally a diluent or carrier.

17. The composition of claim 16, wherein the composition is a lyophilized composition and/or a reconstituted lyophilized composition.

18. A method for absorbing CO₂ from a CO₂-containing effluent or gas, the process comprising: contacting the CO₂-containing effluent or gas with an aqueous absorption solution to dissolve the CO₂ into the aqueous absorption solution; and providing the recombinant carbonic anhydrase polypeptides of claim 1, an isolated nucleic acid encoding the recombinant carbonic anhydrase polypeptide, the vector or expression cassette encoding the isolated nucleic acid, and/or the cell comprising the vector, a composition comprising the recombinant carbonic anhydrase polypeptide, the isolated nucleic acid, the vector or expression cassette, and/or the cell, to catalyze the hydration reaction of the dissolved CO₂ into bicarbonate and hydrogen ions or the reverse reaction.

19. A method of accelerating weathering of minerals or for enhancing carbon storage or sequestration in a water source, optionally an ocean or sea, the method comprising contacting a water source, optionally a mineral rich water source, optionally an ocean or sea, with the recombinant carbonic anhydrase polypeptides of claim 1, an isolated nucleic acid encoding the recombinant carbonic anhydrase polypeptide, the vector or expression cassette encoding the isolated nucleic acid, and/or the cell comprising the vector, a composition comprising the recombinant carbonic anhydrase polypeptide, the isolated nucleic acid, the vector or expression cassette, and/or the cell, optionally such that the recombinant carbonic anhydrase polypeptide catalyzes the reversible hydration of CO₂ to bicarbonate.

20. A kit comprising one or more of the recombinant carbonic anhydrase polypeptides of claim 1, an isolated nucleic acid encoding the recombinant carbonic anhydrase polypeptide, the vector or expression cassette encoding the isolated nucleic acid, and/or the cell comprising the vector, a composition comprising the recombinant carbonic anhydrase polypeptide, the isolated nucleic acid, the vector or expression cassette, and/or the cell; and optionally instructions for use.


SEQUENCE LISTING (18 sequences)

SEQ ID NO: 1 - protein, 219 aa, synthetic construct
HWGYSGETGPENWAKLTPEFGACAGKNQTPVNLDGFIKAELKPLKFNYKAGGSQILNNGHTVQVVYDAGSNVVIDGVEYALKQFHFHAPSENQIKGESYPLEGHFVHADKDGNLAVVTVMFKEGHANEALESLWTHMPAKEGDKILSPANNALKFFPKDHAYYRFSGSLTTPPCTEGVRWIVMKKPVFVSKAQIDAFKKVMVHANNRPLQPINAREILG

SEQ ID NO: 2 - protein, 219 aa, synthetic construct
HWGYSGETGPENWAKLTPEFGACAGKNQTPVNLSGFVKAELKPLKFNYKAGGSQILNNGHTVQVVYDAGSNVVIDGVEYALKQFHFHAPSENQIKGESYPLEGHFVHADKDGNLAVVTVMFKEGHENEALESLWAHMPAKEGDKILSPAFNALKLFPKNHEYYRFSGSLTTPPCTEGVRWIVMKKPVFVSKAQIDAFKKVMGHDNNRPLQPINAREILE

SEQ ID NO: 3 - protein, 219 aa, synthetic construct
HWSYSGETGPEHWAKLTPEFAACAGKNQSPVDISGTVKAELKPLKINYKAAGSEIVNNGHTIQVNYAAGSTLVIDGVEFELKQFHFHAPSENTIKGQSYPLEAHFVHADKDGNLAVVGVMFKEGKENQALEKLWAHMPKEEGEKVLSPAINALALLPKKHEYYRFSGSLTTPPCSEGVRWIVMKKPVSVSKEQIDAFKKIMGHDNNRPLQPINARTILE

SEQ ID NO: 4 - protein, 219 aa, synthetic construct
HWSYSGETGPEHWAKLTPEYFACAGKNQSPVDISGTVKAELKPLKINYKAAGSEIVNNGHTIQVNYAPGSTLVIDGTEFELKQFHFHAPSENTVKGQSYPLEAHFVHADKDGNLAVIGVMFKEGKENQALEKLWANMPKEEGEKVLSPAINALALLPKKHEYYRFSGSLTTPPCSEGVRWIVMKKPVTVSKEQIDAFKKIMGHDNNRPVQPINARVILE

SEQ ID NO: 5 - protein, 219 aa, synthetic construct
HWSYSGETGPEHWGKLKPEYFMCKGKNQSPVDINGTVEAELKPLKINYKAAGSEIVNNGHTIQVNYEPGSYLVVDGIKFELKQFHFHAPSEHTIKGKSYPLEAHFVHADKDGNLAVIGVMFKEGKENPELEKLWKNMPKEEGKKVLSHAINAYALLPKKKKYYRYSGSLTTPPCSEGVRWIVMKKPLTVSKEQIEKFKKIMGHDNNRPVQPINARMILE

SEQ ID NO: 6 - protein, 219 aa, synthetic construct
HWSYEGETGPEHWGKLKPEYFMCKGKNQSPVDINGTVEAELEPLNINYKAAGSEIVNNGHTIQVNYKEDNYLVIDGKKFHLKQFHFHAPSEHTVKGKYYPLEMHFVHKDKDGNLAVIGVMFKEGKENPELEKLWKNAPKEEGKKVLDSSINMNALLPKKKDYYRYSGSLTTPPCSEGVRWIVLKKPITVSKEQIEKFKKIMGHDNNRPVQPINARMILE

SEQ ID NO: 7 - protein, 219 aa, synthetic construct
HWSYEGENGPEHWAKLKPEYFWCKLKNQSPVDISDKVKAKLEKLNINYKKANSEIVNNGHTIQVNVKEDNTLNIKGKKYHLKQFHFHAPSEHTVEGKYYPLEMHFVHKDKDGNIAVIGVMFKEGKANPELDKVFKNAPKEEGEKVLDGSINLNALLPKDKNYYTYSGSLTTPPCTEGVRWIVLKQPITVSKQQIEKFKSIMKHDNNRPVQPINSRYILE

SEQ ID NO: 8 - protein, 219 aa, synthetic construct
HWSYEGENGPENWAKLNPEYFWCNLKNQSPVDISDKVHAKLEKLNINYNKANPEIVNNGHTIQVNVLEDFKLNIKGKEYHLKQFHFHAPSEHTVNGKYYPLEMHLVHKDKDGNIAVIGVFFKEGKANPELDKVFKNALKEEGSKVFDGSINLNALLPPVKNYYTYSGSLTTPPCTEGVLWIVLKQPITASKQQIELFKSIMKHNNNRPTQPINSRYILE

SEQ ID NO: 9 - protein, 219 aa, synthetic construct
HWSYHGETGPEHWGDLKDEYIMCKGKNQSPVDINRIVEAELKKLKINYKSGGSSIVNNGHTIKVSYEPGSYIVVDGIKFELKQFHFHAPSEHKIKGKSYPFEAHFVHADKDGNLAVIGVVFKEGKENPVIEKLWKNLPSEVGKKVLAHKINAYDLLPKKKKYYRYSGSLTTPPCSEGVRWIVMKEELEMSKEQIEKFRKLMGGDTNRPVQPLNARMIME

SEQ ID NO: 10 - protein, 219 aa, synthetic construct
HWSYHGETGPQHWGDLKNEYIMCKGKNQSPVDISRIVEAELKKLKINYSSGGSSITNNGHTIKVSYEPGSYIVVDGIRFELKQFHFHAPSEHKIKGKSYPFEAHFVHADKDGNLAVIGVIFKEGKKNPVIEKIWKNLPSEAGKTVLAHKINAYDLLPKKKKYYRYSGSLTTPPCSEGVRWIVMKEEMELSKEQIEKFRKLMGGDTNRPVQPLNARMIME

SEQ ID NO: 11 - protein, 219 aa, synthetic construct
NWSYHGETGPEHWGDLKDEYIMCKGKNQSPVDINRIVEAELKDLKINYKAGATSIVNNGHTIKVSYEPGSYIVVDGIKFELKQFHFHAPSEHKIKGKSYPFEAHFVHADKDGNLAVIGVVFKEGKENPVIEKLWKNLPSEVGKKVLAHKINAYDLLPKKKKYYRYSGSLTTPPCSEGVRWIVMKEELEMSKEQIEKFRKLMGGDTNRPVQPLNARMIME

SEQ ID NO: 12 - protein, 219 aa, synthetic construct
  VARIANT 1: note = X is H or N
  VARIANT 3: note = X is G or S
  VARIANT 5: note = X is S, H or E
  VARIANT 8: note = X is T or N
  VARIANT 11: note = X is E or Q
  VARIANT 12: note = X is N or H
  VARIANT 14: note = X is A or G
  VARIANT 15: note = X is K or D
  VARIANT 17: note = X is T, N or K
  VARIANT 18: note = X is P, N or D
  VARIANT 20: note = X is F or Y
  VARIANT 21: note = X is G, F, I or A
  VARIANT 22: note = X is A, W or M
  VARIANT 24: note = X is A, N or K
  VARIANT 25: note = X is G or L
  VARIANT 29: note = X is T or S
  VARIANT 32: note = X is N or D
  VARIANT 33: note = X is L or I
  VARIANT 34: note = X is S, D or N
  VARIANT 35: note = X is G, D or R
  VARIANT 36: note = X is F, K, I or T
  VARIANT 37: note = X is V or I
  VARIANT 38: note = X is K, H or E
  VARIANT 40: note = X is E or K
  VARIANT 42: note = X is K or E
  VARIANT 43: note = X is P, K or D
  VARIANT 45: note = X is K or N
  VARIANT 46: note = X is F or I
  VARIANT 49: note = X is K, N or S
  VARIANT 50: note = X is A, K or S
  VARIANT 51: note = X is G or A
  VARIANT 52: note = X is G, N or A
  VARIANT 53: note = X is S, T or P
  VARIANT 54: note = X is Q, E or S
  VARIANT 56: note = X is L, V or T
  VARIANT 62: note = X is V or I
  VARIANT 63: note = X is Q or K
  VARIANT 65: note = X is V, N or S
  VARIANT 66: note = X is Y or V
  VARIANT 67: note = X is D, L, K, E or A
  VARIANT 68: note = X is A, E or P
  VARIANT 69: note = X is G or D
  VARIANT 70: note = X is S, F or N
  VARIANT 71: note = X is N, K, T or Y
  VARIANT 72: note = X is V, L or I
  VARIANT 73: note = X is V or N
  VARIANT 74: note = X is I or V
  VARIANT 75: note = X is D or K
  VARIANT 77: note = X is V, K, I or T
  VARIANT 78: note = X is E, K or R
  VARIANT 79: note = X is Y or F
  VARIANT 80: note = X is A, H or E
  VARIANT 92: note = X is H or N
  VARIANT 93: note = X is T, Q or K
  VARIANT 94: note = X is I or V
  VARIANT 95: note = X is K, N or E
  VARIANT 97: note = X is E, K or Q
  VARIANT 98: note = X is S or Y
  VARIANT 101: note = X is L or F
  VARIANT 103: note = X is G, M or A
  VARIANT 105: note = X is F or L
  VARIANT 108: note = X is A or K
  VARIANT 114: note = X is L or I
  VARIANT 117: note = X is V or I
  VARIANT 118: note = X is T or G
  VARIANT 120: note = X is M, F, I or V
  VARIANT 125: note = X is H or K
  VARIANT 126: note = X is E, K or A
  VARIANT 128: note = X is E, P or Q
  VARIANT 129: note = X is A, E or V
  VARIANT 130: note = X is L or I
  VARIANT 131: note = X is E or D
  VARIANT 132: note = X is S or K
  VARIANT 133: note = X is L, V or I
  VARIANT 134: note = X is W or F
  VARIANT 135: note = X is A, T or K
  VARIANT 136: note = X is H or N
  VARIANT 137: note = X is M, A or L
  VARIANT 138: note = X is P or L
  VARIANT 139: note = X is A, K or S
  VARIANT 140: note = X is K or E
  VARIANT 141: note = X is A, E or V
  VARIANT 143: note = X is D, S, E or K
  VARIANT 144: note = X is K or T
  VARIANT 145: note = X is I or V
  VARIANT 146: note = X is L or F
  VARIANT 147: note = X is S, D or A
  VARIANT 148: note = X is P, H, G or S
  VARIANT 149: note = X is A, S or K
  VARIANT 150: note = X is F, N or I
  VARIANT 152: note = X is A, L or M
  VARIANT 153: note = X is L, N or Y
  VARIANT 154: note = X is K, A or D
  VARIANT 155..156: note = X is L or F
  VARIANT 158: note = X is K or P
  VARIANT 159: note = X is N, D, V or K
  VARIANT 160: note = X is H or K
  VARIANT 161: note = X is E, A, N, K or D
  VARIANT 164: note = X is R or T
  VARIANT 165: note = X is F or Y
  VARIANT 175: note = X is T or S
  VARIANT 179: note = X is R or L
  VARIANT 183: note = X is M or L
  VARIANT 185: note = X is K, Q or E
  VARIANT 186: note = X is P or E
  VARIANT 187: note = X is V, I, M or L
  VARIANT 188: note = X is F, T, E or S
  VARIANT 189: note = X is V, A, L or M
  VARIANT 192: note = X is A, Q or E
  VARIANT 195: note = X is D or E
  VARIANT 196: note = X is A, L or K
  VARIANT 198: note = X is K or R
  VARIANT 199: note = X or K or S
  VARIANT 200: note = X is V, I or L
  VARIANT 202: note = X is G, V or K
  VARIANT 203: note = X is H or G
  VARIANT 204: note = X is D, A or N
  VARIANT 205: note = X is N or T
  VARIANT 209: note = X is L, T or V
  VARIANT 212: note = X is I or L
  VARIANT 214: note = X is A or S
  VARIANT 216: note = X is E, Y, M, V, or T
  VARIANT 218: note = X is L or M
  VARIANT 219: note = X is E or G
XWXYXGEXGPXXWXXLXXEXXXCXXKNQXPVXXXXXXXAXLXXLXXNYXXXXXXIXNNGHTXXVXXXXXXXXXXXGXXXXLKQFHFHAPSEXXXXGXXYPXEXHXVHXDKDGNXAVXXVXFKEGXXNXXXXXXXXXXXXXXGXXXXXXXXNXXXXXPXXXXYYXXSGSLTTPPCXEGVXWIVXKXXXXXSKXQIXXFXXXMXXXXNRPXQPXNXRXIXX

SEQ ID NO: 13 - protein, 254 aa, Sulfurihydrogenibium azorense
MKKFILSILSLSIVSIAGEHAILQKNAEVHHWSYEGENGPENWAKLNPEYFWCNLKNQSPVDISDNYKVHAKLEKLHINYNKAVNPEIVNNGHTIQVNVLEDFKLNIKGKEYHLKQFHFHAPSEHTVNGKYYPLEMHLVHKDKDGNIAVIGVFFKEGKANPELDKVFKNALKEEGSKVFDGSININALLPPVKNYYTYSGSLTTPPCTEGVLWIVLKQPITASKQQIELFKSIMKHNNNRPTQPINSRYILESN

SEQ ID NO: 14 - DNA, 660 bp, synthetic construct
  source 1..660: mol_type = other DNA
cactggtcatatagtggagaaacagggcccgagcattggggtaagcttaagccggaatacttcatgtgcaaaggtaagaaccagagcccggttgacatcaacggcaccgtggaagcagaactcaagccactgaagatcaattataaggcggcaggttcggagatcgtcaataacggccacaccattcaagtaaactacgaaccgggttcctacctggtcgtggacggcattaaattcgagctgaagcagtttcatttccacgcgccaagcgagcacaccatcaagggcaagtcttacccgttggaagcgcattttgttcacgcggataaagacggaaacctggctgtgatcggcgtgatgttcaaagagggcaaagagaatccggaactggagaaactgtggaaaaacatgccgaaggaggaaggtaagaaagttctgagccatgcgatcaacgcttatgctctgttaccgaaaaaaaagaaatattaccgctatagcggttctctgacgactccgccttgtagcgagggtgtgcgttggattgttatgaaaaagccgttgaccgtttccaaagaacaaattgaaaaatttaagaagatcatgggtcacgataataatcgtccggttcagccgattaacgcccgtatgattttggagtaa

SEQ ID NO: 15 - DNA, 660 bp, synthetic construct
  source 1..660: mol_type = other DNA
cactggtcatatgaaggagagacagggcccgagcattggggtaagctcaagccggaatatttcatgtgcaaaggtaagaaccagtcgccggttgatatcaatggcaccgtggaagcggagttggagccgttgaatattaactacaaagcggcgggttctgagatcgttaataacggccataccattcaagtgaactataaagaggacaactacctggttatcgacggcaagaagttccacctgaagcaatttcacttccacgctccgagcgagcacactgtaaaaggcaagtattacccgctggaaatgcactttgttcataaagacaaagatggtaatctggcagttattggtgtgatgtttaaagaaggcaaggaaaatccggaattagagaagctttggaaaaacgcaccgaaagaggaaggtaaaaaggttctggatagcagcatcaacatgaatgcgttgctgccgaaaaagaaggactactaccgctatagcggttccctgaccacgccaccgtgttccgagggcgtgcgttggattgtgctgaagaaaccgattaccgtcagcaaagagcagattgaaaaattcaaaaagatcatgggtcatgataacaaccgtccggtccagcctatcaacgcccgtatgatcctggaataa

SEQ ID NO: 16 - DNA, 660 bp, synthetic construct
  source 1..660: mol_type = other DNA
cactggtcatatgagggagaaaatgggcccgagcattgggcaaaattgaagccggagtacttctggtgtaaattgaagaaccagagcccggtcgacatcagcgataaggtgaaagccaaattggagaagttaaatatcaactataaaaaggcgaattccgagatcgtgaataacggccatacgattcaggttaatgttaaagaagataataccctgaacatcaaaggtaagaaataccatctgaagcaatttcacttccacgctccgagcgagcacacggttgaaggcaaatattacccgctggaaatgcatttcgtgcacaaagacaaggacggtaatattgctgttattggtgttatgtttaaggagggcaaagcgaacccggaactggataaggtctttaaaaacgcgccgaaagaggagggtgaaaaagtgctggacggctccatcaacctcaatgcgctgctgccgaaagacaagaactactacacctatagcggttctctgaccactccgccttgcaccgaaggcgtgcgttggattgttcttaagcaaccgatcaccgtgagcaaacaacagattgaaaagttcaagtcgatcatgaagcacgataacaaccgcccagttcagccgattaactctcgttatatcctggaataa

SEQ ID NO: 17 - DNA, 381 bp, synthetic construct
  source 1..381: mol_type = other DNA
agatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgcggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttg

SEQ ID NO: 18 - protein, 58 aa, synthetic construct
MGSSHHHHHHSSGLVPRGSHMASMTGGEEMGRGSEFELRREACGRTRAPPPPPLRSGC